(19) United States
(12) Patent Application Publication
(10) Pub. No.: US 2011/0277182 A1
(43) Pub. Date: Nov. 10, 2011

(54) MAIZE ACC SYNTHASE 3 GENE AND PROTEIN AND USES THEREOF

(75) Inventors: Xiaoming Bao, Johnston, IA (US); Stephen M. Allen, Wilmington, DE (US)

(73) Assignees: E.I. DUPONT DE NEMOURS AND COMPANY, Wilmington, DE (US); PIONEER HI-BRED INTERNATIONAL, INC., Johnston, IA (US)

(21) Appl. No.: 13/075,260

(22) Filed: Mar. 30, 2011

Related U.S. Application Data
(60) Provisional application No. 61/332,069, filed on May 6, 2010.

(51) Int. Cl. C12N 15/82; C07H 21/00; A01H 5/00; C12N 9/88

(52) U.S. Cl. 800/278; 435/232; 536/23.2; 435/320.1; 800/298; 800/320.1; 800/320.3; 800/320.2; 800/320

(57) ABSTRACT

Methods and compositions for modulating plant development are provided. Nucleotide sequences and amino acid sequences encoding ACC Synthase 3 (ACS3) proteins are provided. The sequences can be used in a variety of methods including modulating development, modulating response to stress, and modulating stress tolerance of a plant. Transformed plants, plant cells, tissues, and seed are also provided.

### CROSS-REFERENCE

This application claims priority to, and hereby incorporates by reference, U.S. Provisional Patent Application Ser. No. 61/332,069 filed May 6, 2010, and U.S. patent application Ser. No. 12/897,489 filed Oct. 4, 2010.

### FIELD OF THE INVENTION

The invention relates to the field of the genetic manipulation of plants, particularly the modulation of gene activity and development in plants.

### BACKGROUND OF THE INVENTION

ACC synthase (ACS) catalyzes the synthesis of 1-aminocyclopropane-1-carboxylic acid (ACC) from S-adenosyl-L-methionine (SAM), the first committed step of ethylene biosynthesis. This step is rate-limiting for ethylene formation; expression of ACS is tightly regulated at both the transcriptional and post-transcriptional levels. (Wang, et al., (2004) Nature 428(6986):945-950, Christians, et al., (2009) Plant Journal 57(2):332-345).

### BRIEF SUMMARY OF THE INVENTION

In certain embodiments the present invention provides a previously unknown maize ACC synthase, designated ZmACS3. Modulation of expression of ZmACS3, particularly downregulation of ZmACS3, alone or in combination with modulation of other genes, can reduce ethylene production, resulting in increased growth rate and improved stress tolerance in plants. For example, suppression of expression of both ZmACS6 and ZmACS3 in maize may result in higher growth rate and improved yield under optimal and/or stress (e.g., drought) conditions.

Certain compositions of the invention include an isolated polynucleotide selected from the group consisting of: (a) a polynucleotide comprising SEQ ID NO:1 or 2; (b) a polynucleotide encoding the amino acid sequence of SEQ ID NO: 3; (c) a polynucleotide having at least 90% sequence identity to SEQ ID NO: 2, wherein the polynucleotide encodes a polypeptide having ACC synthase activity; (d) a polynucleotide that hybridizes under stringent conditions to the complement of the polynucleotide of (a), wherein the stringent conditions comprise 50% formamide, 1 M NaCl, 1% SDS at 37° C. and a wash in 0.1×SSC at 60° C. to 65° C.; (f) a subsequence of at least about 25 nucleotides of SEQ ID NO: 1 or SEQ ID NO: 2, which subsequence functions to suppress expression of one or more ACS genes; (g) a subsequence of any of (a), (b), (c), (d) or (e). Nucleotide constructs comprising the polynucleotide of the invention are also provided.

Methods and compositions are provided to modulate plant development using DNA, RNA or protein representing or derived from the ZmACS3 gene. Certain embodiments provide an isolated polypeptide comprising an amino acid sequence selected from the group consisting of: (a) the polypeptide comprising the amino acid sequence of SEQ ID NO: 3; (b) a polypeptide having at least 90% sequence identity to the full length of SEQ ID NO: 3, wherein the polypeptide has ACC synthase activity; (c) a polypeptide encoded by a polynucleotide that hybridizes under stringent conditions to a polynucleotide comprising the complement of SEQ ID NO: 2, wherein the stringent conditions comprise 50% formamide, 1 M NaCl, 1% SDS at 37° C. and a wash in 0.1×SSC at 60° C. to 65° C. and (d) a polypeptide having at least 70 consecutive amino acids of SEQ ID NO:3, wherein the polypeptide retains ACC synthase activity.

Certain embodiments of the invention include plants having a transgene comprising a polynucleotide of the invention operably linked to a heterologous promoter that drives expression in the plant. Expression of the transgene may be constitutive, or may be directed preferentially to a particular plant cell type or plant tissue type and/or phase of plant growth, or may be inducible or otherwise controlled. Methods are provided to modulate plant growth and development, particularly plant response to stress, particularly abiotic stress, relative to a control plant, control plant cell or control plant part. The modulated growth or development may be reflected in, for example, higher growth rate, higher yield, altered morphology or appearance and/or an altered response to stress including an improved tolerance to stress. In certain embodiments, the stress is cold, salt or drought. In certain embodiments, yield is increased or maintained during periods of abiotic stress, relative to yield of a control plant. Yield may be measured, for example, in terms of seed yield, plant biomass yield or recovery of other plant product or products. Seed set may be measured by, for example, seed number, total seed mass, average seed mass or some combination of these or other measures.

### BRIEF DESCRIPTION OF THE DRAWINGS

FIG. 1 provides an alignment of the amino acid sequences of ZmACS6 (SEQ ID NO: 4) and ZmACS3 (SEQ ID NO: 3).

FIG. 2 provides an alignment of the cDNA sequences of ZmACS6 (SEQ ID NO: 6) and ZmACS3 (SEQ ID NO: 2).

FIG. 3 provides an alignment of a segment (TR4; SEQ ID NO: 5) of a hairpin downregulation construct with a portion of the ACS3 cDNA (portion of SEQ ID NO: 2).

FIG. 4 is a schematic of an expression cassette for which sequence is provided in SEQ ID NO: 7.

FIG. 5 shows expression levels of ACS3 in flooded root tissues.

FIG. 6 shows expression levels of ACS6 in flooded root tissues.

FIG. 7 is provided as an example of qRT-PCR results.

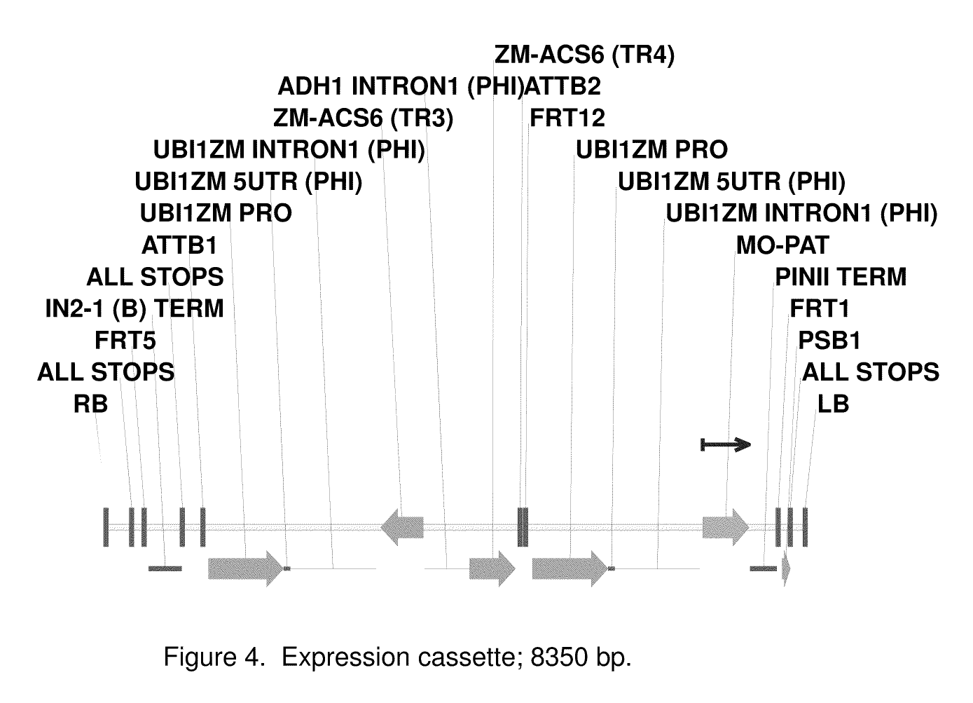

TABLE 1
Sequence Listing Summary

| SEQ ID NO: | |
|---|---|
| 1 | ZmACS3 genomic |
| 2 | ZmACS3 cDNA |
| 3 | ZmACS3 amino acid |
| 4 | ZmACS6 amino acid |
| 5 | hpRNA component |
| 6 | ZmACS6 cDNA |
| 7 | Expression cassette of FIG. 4 |
| 8 | primer |
| 9 | primer |
| 10 | MGB probe |
| 11 | PnACS3 cDNA |
| 12 | PnACS3 amino acid |

### DETAILED DESCRIPTION OF THE INVENTION

The present invention now will be described more fully in text and accompanying drawings, in which some, but not all, embodiments of the invention are shown. Indeed, the invention may be embodied in many different forms and should not be construed as limited to the embodiments set forth herein. Many modifications and other embodiments of the invention set forth herein will come to mind to one skilled in the art to which this invention pertains, having the benefit of the teachings presented in the accompanying descriptions and the associated drawings. Therefore, it is to be understood that the invention is not to be limited to the specific embodiments disclosed and that modifications and other embodiments are intended to be included within the scope of the appended claims. It is also to be understood that the terminology used herein is for the purpose of describing particular embodiments only, and is not intended to be limiting. Although specific terms are employed herein, they are used in a generic and descriptive sense only and not for purposes of limitation.

The article “a” and “an” may be used herein to refer to one or more than one (i.e., to at least one) of the grammatical object of the article. Thus, for example, reference to “a cell” includes a combination of two or more cells, and the like.

### DEFINITIONS

Unless defined otherwise, all technical and scientific terms used herein have the same meaning as commonly understood by one of ordinary skill in the art to which the invention pertains. In describing and claiming the present invention, the following terminology will be used in accordance with the definitions set out below.

A “control” or “control plant” or “control plant cell” provides a reference point for measuring changes in phenotype of a subject plant or plant cell in which genetic alteration, such as transformation, has been effected as to a gene of interest. A subject plant or plant cell may be descended from a plant or cell so altered and will comprise the alteration.

A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest or (e) the subject plant or plant cell itself, under conditions in which the gene of interest is not expressed.

A polynucleotide sequence is said to “encode” a sense or antisense RNA molecule or RNA silencing or interference molecule, or a polypeptide, if the polynucleotide sequence can be transcribed (in spliced or unspliced form) and/or translated into the RNA or polypeptide or a subsequence thereof.

The term “endogenous” relates to any nucleic acid sequence or amino acid sequence that is already present in a cell. Typically an endogenous sequence is native to a non-transgenic plant; however, in certain instances, e.g. gene stacking, “endogenous” may refer to a sequence introduced by a prior transformation process. See also “host cell.”

An “expression cassette” is a nucleic acid construct typically including expression control (regulatory) sequences, such as a promoter and/or terminator and structural sequences comprising a polynucleotide. The polynucleotide may encode a polypeptide. A polynucleotide which does not encode a polypeptide may provide an alternate function, e.g., in a downregulation system as known in the art or described elsewhere herein. An expression cassette may be part of a vector, such as a plasmid, a viral vector, etc., capable of producing transcripts and, potentially, polypeptides encoded by a polynucleotide sequence. An expression vector is capable of producing transcripts in a cell, e.g., a bacterial cell, or a plant cell, in vivo or in vitro. Expression of a product in a cell in vitro can be constitutive or inducible, depending, e.g., on the promoter selected. Expression of a product in a plant cell can be constitutive, inducible, tissue-specific, tissue-preferred, organ-preferred or otherwise limited. Antisense, sense or RNA interference or silencing configurations that are not, or cannot be, translated are expressly included by this definition. In the context of an expression vector, a promoter is said to be “operably linked” to a polynucleotide sequence if it is capable of regulating expression of the associated polynucleotide sequence. The term also applies to alternative exogenous gene constructs, such as expressed or integrated transgenes. Similarly, the term “operably linked” applies equally to alternative or additional transcriptional regulatory sequences, such as enhancers, associated with a polynucleotide sequence.

“Expression of a gene” or “expression of a nucleic acid” means transcription of DNA into RNA (optionally including modification of the RNA, e.g., splicing) or translation of RNA into a polypeptide (possibly including subsequent modification of the polypeptide, e.g., posttranslational modification) or both transcription and translation, as indicated by the context.

The term “gene” is used broadly to refer to any nucleic acid associated with a biological function. Genes typically include coding sequences and/or the regulatory sequences required for expression of such coding sequences. The term “gene” applies to a specific genomic sequence, as well as to a cDNA or an mRNA encoded by that genomic sequence. Genes also include non-expressed nucleic acid segments that, for example, form recognition sequences for other proteins. Non-expressed regulatory sequences include promoters and enhancers, to which regulatory proteins such as transcription factors bind, resulting in transcription of adjacent or nearby sequences.

The term “gene product” includes mRNA, polypeptide, and/or protein encoded by a nucleotide sequence. For example, a gene product may be a complete and functional protein sequence, a partial protein sequence, a complete processed or unprocessed RNA, or a partial RNA, such as one which impacts stability or translation of a homologous RNA.

As used herein, a “heterologous” nucleic acid is a nucleic acid that originates from a foreign species, or, if from the same species, is substantially modified from its native form in composition and/or genomic locus, by deliberate human intervention. For example, a promoter operably linked to a heterologous structural gene is from a species different from that from which the structural gene was derived, or, if from the same species, one or both are substantially modified from their original form and/or genomic location.

A “host cell”, as used herein, is a cell which has been transformed or transfected or is capable of transformation or transfection, by an exogenous polynucleotide sequence. “Exogenous polynucleotide sequence” is defined to mean a sequence not naturally in the cell, or which is naturally present in the cell but at a different genetic locus, in different copy number or under direction of a different regulatory element. Host cells may be prokaryotic cells such as E. coli or eukaryotic cells such as yeast, insect, amphibian or mammalian cells. Optimally, host cells are monocotyledonous or dicotyledonous plant cells. A particularly optimal monocotyledonous host cell is a maize host cell.

In the context of the invention, the term “isolated” refers to a biological material, such as a nucleic acid or a protein, which is substantially free from components that normally accompany or interact with it in its naturally occurring environment. The isolated material optionally comprises material not found with the material in its natural environment, e.g., a cell. For example, if the material is in its natural environment, such as a cell, the material has been placed at a location in the cell (e.g., genome or genetic element) not native for that material. For example, a naturally occurring nucleic acid (e.g., a coding sequence, a promoter, or an enhancer) becomes isolated if it is introduced by non-naturally occurring means to a locus of the genome (e.g., a vector, such as a plasmid or virus vector or amplicon) not native to that nucleic acid. An isolated plant cell, for example, can be in an environment (e.g., a cell culture system, or purified from cell culture) other than the native environment of wild-type plant cells (e.g., a whole plant).

The term “nucleic acid” or “polynucleotide” is generally used in its art-recognized meaning to refer to a ribose nucleic acid (RNA) or deoxyribose nucleic acid (DNA) polymer or analog thereof, e.g., a nucleotide polymer comprising modifications of the nucleotides, a peptide nucleic acid or the like. In certain applications, the nucleic acid can be a polymer that includes multiple monomer types, e.g., both RNA and DNA subunits. A nucleic acid can be, e.g., a chromosome or chromosomal segment, a vector (e.g., an expression vector), an expression cassette, a naked DNA or RNA polymer, the product of a polymerase chain reaction (PCR), an oligonucleotide, a probe, etc. A nucleic acid can be, e.g., single-stranded and/or double-stranded. Unless otherwise indicated, a particular nucleic acid sequence of this invention optionally comprises or encodes complementary sequences, in addition to any sequence explicitly indicated.

A “phenotype” is the display of a trait in an individual plant resulting from the interaction of gene expression and the environment.

The term “plant” refers generically, within the appropriate context, to any of: whole plants, plant parts or organs (e.g., leaves, stems, roots), vegetative organs/structures (e.g. leaves, stems, tubers), flowers and floral organs/structures (e.g. bracts, sepals, petals, stamens, carpels, anthers, ovules), seed (including, e.g., embryo, endosperm, seed coat), fruit (the mature ovary), plant tissue (e.g. vascular tissue), tissue culture callus, plant cells (e.g. guard cells, egg cells, mesophyll cells) and progeny of same. Plant cell, as used herein, further includes, without limitation, cells obtained from or found in: seeds, cultures, suspension cultures, embryos, meristematic regions, callus tissue, leaves, roots, shoots, gametophytes, sporophytes, pollen and microspores. Plant cells can also be understood to include modified cells, such as protoplasts, obtained from the aforementioned tissues. As used herein, “grain” is intended the mature seed produced by commercial growers for purposes other than growing or reproducing the species. Progeny, variants and mutants of the regenerated plants are also included within the scope of the invention, provided that these parts comprise the introduced nucleic acid sequences.

The term “polynucleotide sequence” or “nucleotide sequence” refers to a contiguous sequence of nucleotides in a single nucleic acid or to a representation, e.g., a character string, thereof. That is, a “polynucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

A “polypeptide” is a polymer comprising two or more amino acid residues (e.g., a peptide or a protein). The polymer can additionally comprise non-amino acid elements such as labels, quenchers, blocking groups or the like and can optionally comprise modifications such as glycosylation or the like. The amino acid residues of the polypeptide can be natural or non-natural and can be unsubstituted, unmodified, substituted or modified.

A “promoter”, as used herein, includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells, such as Agrobacterium or Rhizobium. Examples of promoters under developmental control include promoters that preferentially initiate transcription in certain tissues or in certain regions, such as leaves, roots, seeds, endosperm, embryo or meristematic regions. Such promoters are referred to as “tissue-preferred” or “tissue-specific”. A temporally-regulated promoter drives expression at particular times, such as between 0 and 25 days after pollination. A “cell-type-preferred” promoter primarily drives expression in certain cell types in one or more organs, for example, vascular cells in roots and/or leaves. An “inducible” promoter is a promoter that is under environmental control and may be inducible or de-repressible. Examples of environmental conditions that may effect transcription by inducible promoters include anaerobic conditions, the presence of light or the presence of a chemical. Tissue-specific, cell-type-specific and inducible promoters are “non-constitutive” promoters. A “constitutive” promoter is a promoter that is active under most environmental conditions and in all or nearly all tissues, at all or nearly all stages of development.

The term “recombinant” indicates that the material (e.g., a cell, a nucleic acid or a protein) has been artificially or synthetically (non-naturally) altered by human intervention. The alteration can be performed on the material within or removed from, its natural environment or state. For example, a “recombinant nucleic acid” is one that is made by recombining nucleic acids, e.g., during cloning, DNA shuffling or other procedures; a “recombinant polypeptide” or “recombinant protein” may be a polypeptide or protein which is produced by expression of a recombinant nucleic acid. Examples of recombinant cells include cells containing recombinant nucleic acids and/or recombinant polypeptides.

In certain embodiments the nucleic acid sequences of the present invention can be combined, or “stacked,” with any combination of polynucleotide sequences of interest in order to create a plant or plant cell with a desired phenotype, which phenotype may reflect various traits. The combinations generated may include multiple copies of any of the polynucleotides of interest. For example, a polynucleotide of the present invention may be stacked with any other polynucleotide(s) of the present invention and/or with polynucleotides which are of interest for their effect on the same trait or a different trait.

A “subject plant or plant cell” is one in which an alteration, such as transformation or introduction of a polypeptide, has occurred or is a plant or plant cell which is descended from a plant or cell so altered and which comprises the alteration.

The term “subsequence” or “fragment” means any portion of an entire sequence.

“Transformation”, as used herein, is the process by which a cell is “transformed” by exogenous DNA when such exogenous DNA has been introduced inside the cell membrane. Exogenous DNA may or may not be integrated (covalently linked) into chromosomal DNA making up the genome of the cell. In prokaryotes and yeasts, for example, the exogenous DNA may be maintained on an episomal element, such as a plasmid. With respect to higher eukaryotic cells, a stably transformed or transfected cell is one in which the exogenous DNA has become integrated into the chromosome so that it is inherited by daughter cells through chromosome replication. This stability is demonstrated by the ability of the eukaryotic cell to establish cell lines or clones comprised of a population of daughter cells containing the exogenous DNA.

The term “transgenic” refers to a plant into which one or more nucleic acid sequences has been introduced by a transformation process or to a plant which is descended from such a plant and which retains said introduced nucleic acid. The introduced nucleic acid may be transiently or stably incorporated within the plant. See also, “transformation.”

A “transposable element” (TE) or “transposable genetic element” is a DNA sequence that can move from one location within the genome of a cell. Movement of a transposable element can occur from episome to episome, from episome to chromosome, from chromosome to chromosome or from chromosome to episome. Transposable elements are characterized by the presence of inverted repeat sequences at their termini. Mobilization is mediated enzymatically by a “transposase.” Structurally, a transposable element is categorized as a “transposon” (“TN”) or an “insertion sequence element” (IS element) based on the presence or absence, respectively, of genetic sequences in addition to those necessary for mobilization of the element. A mini-transposon or mini-IS element typically lacks sequences encoding a transposase.

The term “variant” with respect to a polypeptide refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variation can also include amino acid deletion or insertion, or both. Guidance in determining which amino acid residues can be substituted, inserted or deleted without eliminating biological or immunological activity can be found using computer programs well known in the art, for example, DNASTAR software. Examples of conservative substitutions are also described below.

The term “vector” refers to the means by which a nucleic acid can be propagated and/or transferred between organisms, cells or cellular components. Vectors include plasmids, viruses, bacteriophage, pro-viruses, phagemids, transposons and artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not autonomously replicating.

### Compositions

Compositions of the invention include polynucleotide and amino acid sequences of ACS3 that are involved in regulating plant growth and development. In some embodiments, the present invention provides for isolated nucleic acid molecules comprising nucleotide sequences encoding the amino acid sequence shown in SEQ ID NO: 3. Further provided are polypeptides having an amino acid sequence encoded by a nucleic acid molecule (SEQ ID NO: 1 or 2) described herein and fragments and variants thereof.

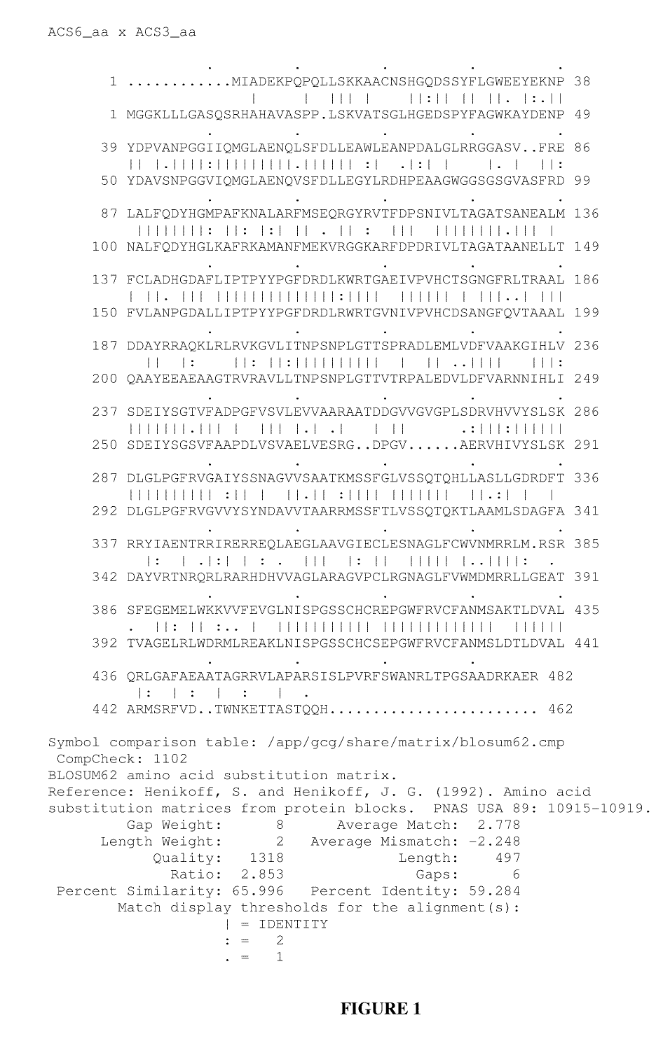

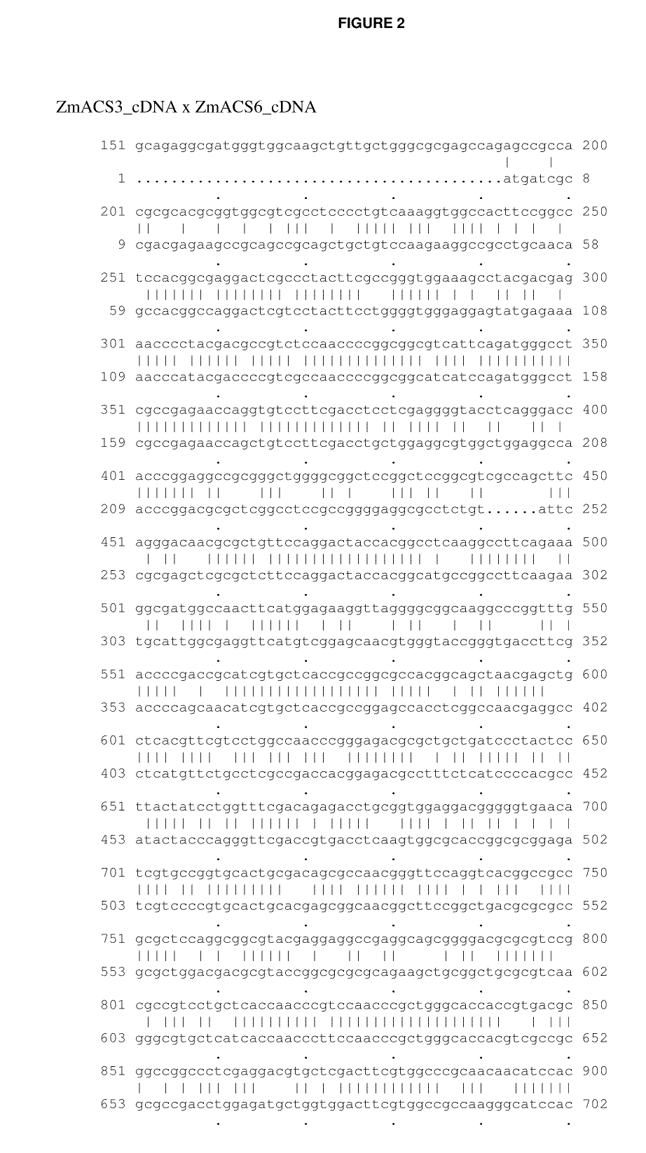

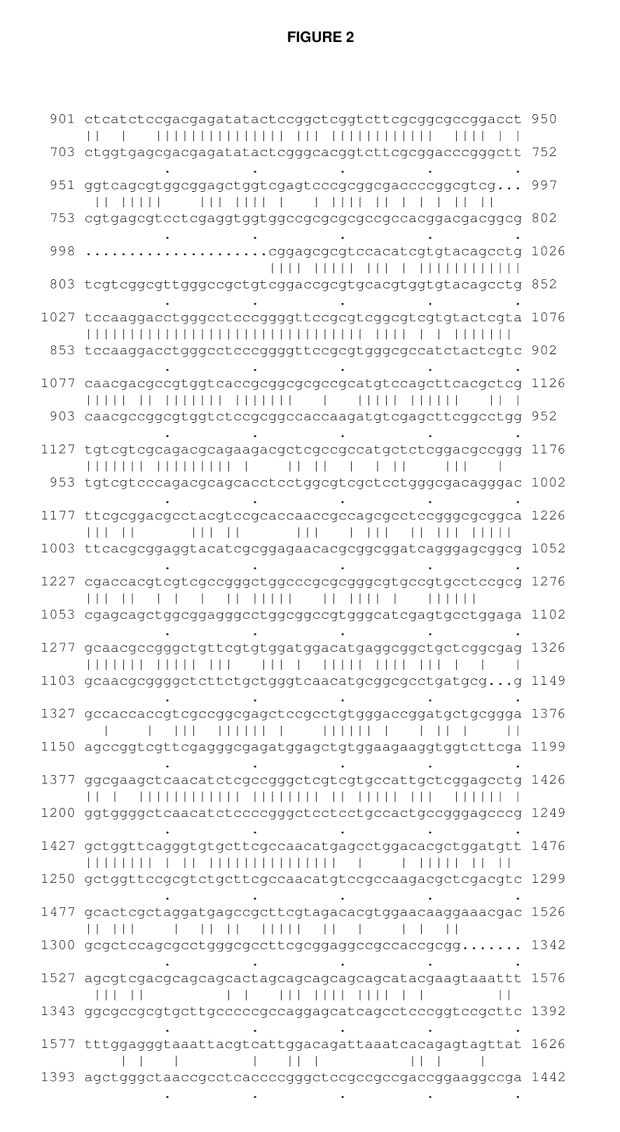

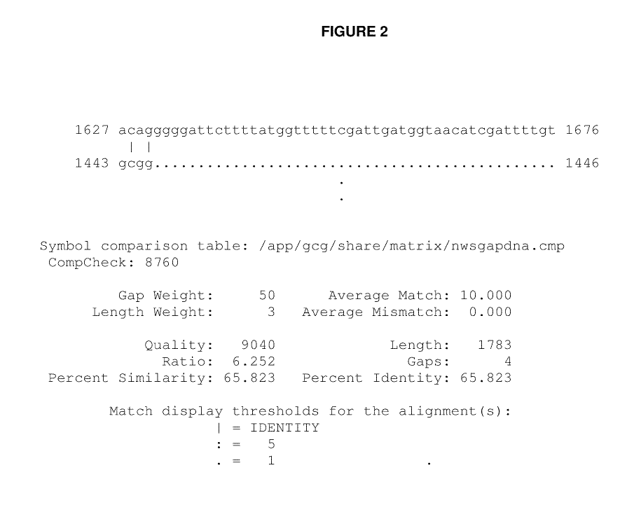

The ACS3 polypeptide shares moderate (59%) identity with the ACS6 protein; see, FIG. 1 for an alignment. Identity between the cDNA sequences of ACS6 and ACS3 is approximately 66%; see, FIG. 2 for an alignment. Thus downregulation constructs are designed that can affect both ACS6 and ACS3, or affect one and not the other, by judicious selection of RNAi target sequences based on the alignment and identities shown herein.

Further, phenotype conferred by the ACS6 and/or ACS3 gene may vary due to the potential for interaction and/or overlapping function of ACS6 and ACS3 gene products. Both enzyme levels, and relative level of each enzyme to the other, as well as to other ACC synthases, may be useful in improving tolerance to drought or other abiotic stress, or any other advantageous phenotype such as increase in yield.

The invention encompasses isolated or substantially purified nucleic acid or protein compositions. An “isolated” or “purified” nucleic acid molecule or protein or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material or culture medium when produced by recombinant techniques or substantially free of chemical precursors or other chemicals when chemically synthesized. Optimally, an “isolated” nucleic acid is free of sequences (optimally protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5% or 1% (by dry weight) of contaminating protein. When the protein of the invention or biologically active portion thereof is recombinantly produced, optimally culture medium represents less than about 30%, 20%, 10%, 5% or 1% (by dry weight) of chemical precursors or non-protein-of-interest chemicals.

Fragments and variants of the disclosed nucleotide sequences and proteins encoded thereby are also encompassed by the present invention. By “fragment” is intended a portion of the nucleotide sequence or a portion of the amino acid sequence and hence protein encoded thereby. Fragments of a nucleotide sequence may encode protein fragments that retain the biological activity of the native protein and hence have ACS3 activity. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides and up to the full-length nucleotide sequence encoding the polypeptide of the invention.

By “ACS3 activity” or “ACC Synthase 3 activity” is intended the ACS3 polypeptide has exemplary activity, such as in catalyzing a step in ethylene synthesis. Methods to assay for such activity are known in the art and are described more fully below. Depending on context, “ACS3 activity” may refer to the activity of a native ACS3 polynucleotide or polypeptide. Such native activity may be modulated by expression of a heterologous ZmACS3 sequence as provided herein, for example when provided in a construct designed for downregulation of the native ZmACS3.

A fragment of an ACS3 nucleotide sequence that encodes a biologically active portion of an ACS3 protein of the invention will encode at least 15, 25, 30, 50, 100, 150, 200, 250, 300, 350, 400 or 450, contiguous amino acids or up to the total number of amino acids present in a full-length ACS3 protein of the invention. Fragments of an ACS3 nucleotide sequence that are useful as hybridization probes or PCR primers or in downregulation constructs generally need not encode a biologically active portion of an ACS3 protein.

Thus, a fragment of an ACS3 nucleotide sequence may encode a biologically active portion of an ACS3 protein or it may be a fragment that can be used as a hybridization probe or PCR primer or in downregulation using methods disclosed herein or known in the art. A biologically active portion of an ACS3 protein can be prepared by isolating a portion of an ACS3 nucleotide sequences of the invention, expressing the encoded portion of the ACS3 protein (e.g., by recombinant expression in vitro) and assessing the activity of the encoded portion of the ACS3 protein. Nucleic acid molecules that are fragments of an ACS3 nucleotide sequence comprise at least 16, 20, 50, 75, 100, 150, 200, 250, 300, 350, 400, 450, 500, 550, 600, 650, 700, 800, 900, 1,000, 1,100, 1,200, 1,300, 1,400 or 1,500 contiguous nucleotides or up to the number of nucleotides present in a full-length ACS3 nucleotide sequence disclosed herein.

“Variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of an ACS3 polypeptide of the invention. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode an ACS3 protein of the invention, or which still function to effect ACS downregulation. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein or known in the art.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, an isolated polynucleotide that encodes a polypeptide with a given percent sequence identity to the polypeptide of SEQ ID NO: 3 is disclosed. Percent sequence identity between any two polypeptides can be calculated using sequence alignment programs and parameters described elsewhere herein or known in the art. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98% or 99% sequence identity.

“Variant” protein is intended to mean a protein derived from the native protein by deletion or addition of one or more amino acids at one or more internal sites in the native protein and/or substitution of one or more amino acids at one or more sites in the native protein. Certain variant proteins encompassed by the present invention are biologically active, that is they continue to possess the desired biological activity of the native protein, that is, the polypeptide has ACS3 activity as described herein. Such variants may result from, for example, genetic polymorphism or from human manipulation. Biologically active variants of a native ACS3 protein of the invention will have at least about 40%, 45%, 50%, 55%, 60%, 65%, 70%, 75%, 80%, 85%, 90%, 91%, 92%, 93%, 94%, 95%, 96%, 97%, 98% or 99% sequence identity to the amino acid sequence for the native protein as determined by sequence alignment programs and parameters described elsewhere herein or known in the art. A biologically active variant of a protein of the invention may differ from that protein by as few as 1-15 amino acid residues, as few as 1-10, such as 6-10, as few as 5, as few as 4, 3, 2 or even 1 amino acid residue.

The proteins of the invention may be altered in various ways including amino acid substitutions, deletions, truncations, and insertions. Methods for such manipulations are generally known in the art. For example, amino acid sequence variants of the ACS3 protein can be prepared by mutations in the DNA. Methods for mutagenesis and nucleotide sequence alterations are well known in the art. See, for example, Kunkel, (1985) Proc. Natl. Acad. Sci. USA 82:488-492; Kunkel, et al., (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff, et al., (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and nucleotide sequences of the invention include both the naturally occurring sequences as well as mutant forms. Likewise, the proteins of the invention encompass both naturally-occurring proteins as well as variations and modified forms thereof. Such variants may continue to possess the desired ACS3 activity. Obviously, for functional proteins, the mutations that will be made in the DNA encoding the variant must not place the sequence out of reading frame and optimally will not create complementary regions that could produce secondary mRNA structure. See, EP Patent Application Publication Number 75,444.

The deletions, insertions and substitutions of the protein sequences encompassed herein are not expected to produce radical changes in the characteristics of the protein. However, when it is difficult to predict the exact effect of the substitution, deletion or insertion in advance of doing so, one skilled in the art will appreciate that the effect will be evaluated by routine screening assays. Various methods for screening for ACS3 activity are provided herein or known in the art.

Variant nucleotide sequences and proteins also encompass sequences and proteins derived from a mutagenic and recombinogenic procedure such as DNA shuffling. With such a procedure, one or more different ACS3 coding sequences can be manipulated to create a new ACS3 possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the ACS3 gene of the invention and other known ACS3 genes to obtain a new gene coding for a protein with an improved property of interest, such as an increased Kₘ in the case of an enzyme. Strategies for such DNA shuffling are known in the art. See, for example, Stemmer, (1994) Proc. Natl. Acad. Sci. USA 91:10747-10751; Stemmer, (1994) Nature 370:389-391; Crameri, et al., (1997) Nature Biotech. 15:436-438; Moore, et al., (1997) J. Mol. Biol. 272:336-347; Zhang, et al., (1997) Proc. Natl. Acad. Sci. USA 94:4504-4509; Crameri, et al., (1998) Nature 391:288-291 and U.S. Pat. Nos. 5,605,793 and 5,837,458.

The nucleotide sequences of the invention can be used to isolate corresponding sequences from other organisms, particularly other plants including other monocots. In this manner, methods such as PCR, hybridization and the like can be used to identify such sequences based on their sequence homology to the sequence set forth herein. Sequences isolated based on their sequence identity to the entire ACS3 sequence set forth herein or to fragments thereof are encompassed by the present invention. Such sequences include sequences that are orthologs of the disclosed sequences. By “orthologs” is intended genes derived from a common ancestral gene and which are found in different species as a result of speciation. Genes found in different species are considered orthologs when their nucleotide sequences and/or their encoded protein sequences share substantial identity as defined elsewhere herein. Functions of orthologs are often highly conserved among species. Thus, isolated sequences that encode for an ACS3 protein and which hybridize under stringent conditions to the ACS3 sequence disclosed herein, or to fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook, et al., (1989) Molecular Cloning: A Laboratory Manual (2d ed., Cold Spring Harbor Laboratory Press, Plainview, N.Y.). See also, Innis et al., eds. (1990) PCR Protocols: A Guide to Methods and Applications (Academic Press, New York); Innis and Gelfand, eds. (1995) PCR Strategies (Academic Press, New York) and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers and the like. For a review of quantitative PCR, see. VanGuilder, et al., (2008) BioTechniques 44:619-626.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments or other oligonucleotides and may be labeled with a detectable group such as ³²P, or any other detectable marker. Thus, for example, probes for hybridization can be made by labeling synthetic oligonucleotides based on the ACS3 sequences of the invention. Methods for preparation of probes for hybridization and for construction of cDNA and genomic libraries are generally known in the art and are disclosed in Sambrook, et al., (1989) Molecular Cloning: A Laboratory Manual (2d ed., Cold Spring Harbor Laboratory Press, Plainview, N.Y.).

For example, the entire ACS3 sequence disclosed herein, or one or more portions thereof, may be used as a probe capable of specifically hybridizing to corresponding ACS3 sequences and messenger RNAs. To achieve specific hybridization under a variety of conditions, such probes include sequences that are unique among ACS3 sequences and are optimally at least about 10 nucleotides in length, and at least about 20 nucleotides in length. Such probes may be used to amplify corresponding ACS3 sequences from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook, et al., (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C. and a wash in 0.5× to 1×SSC at 55 to 60° C. Exemplary high stringency conditions include hybridization in 50% formamide, 1 M NaCl, 1% SDS at 37° C. and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl, (1984) Anal. Biochem. 138:267-284: Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen, (1993) Laboratory Techniques in Biochemistry and Molecular Biology—Hybridization with Nucleic Acid Probes, Part I, Chapter 2 (Elsevier, New York) and Ausubel, et al., eds. (1995) Current Protocols in Molecular Biology, Chapter 2 (Greene Publishing and Wiley-Interscience, New York). See, Sambrook, et al., (1989) Molecular Cloning: A Laboratory Manual (2d ed., Cold Spring Harbor Laboratory Press, Plainview, N.Y.).

The following terms are used to describe the sequence relationships between two or more nucleic acids or polynucleotides: (a) “reference sequence”, (b) “comparison window”, (c) “sequence identity”, (d) “percentage of sequence identity” and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller, (1988) CABIOS 4:11-17; the local alignment algorithm of Smith, et al., (1981) Adv. Appl. Math. 2:482; the global alignment algorithm of Needleman and Wunsch, (1970) J. Mol. Biol. 48:443-453; the search-for-local alignment method of Pearson and Lipman, (1988) Proc. Natl. Acad. Sci. 85:2444-2448; the algorithm of Karlin and Altschul, (1990) Proc. Natl. Acad. Sci. USA 872264, modified as in Karlin and Altschul, (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA and TFASTA in the GCG® Wisconsin Genetics Software Package®, Version 10 (available from Accelrys® Inc., 9685 Scranton Road, San Diego, Calif., USA). Alignments using these programs can be performed using the default parameters. The CLUSTAL program is well described by Higgins, et al., (1988) Gene 73:237-244 (1988); Higgins, et al., (1989) CABIOS 5:151-153; Corpet, et al., (1988) Nucleic Acids Res. 16:10881-90; Huang, et al., (1992) CABIOS 8:155-65 and Pearson, et al., (1994) Meth. Mol. Biol. 24:307-331. The ALIGN program is based on the algorithm of Myers and Miller, (1988) supra. A PAM120 weight residue table, a gap length penalty of 12 and a gap penalty of 4 can be used with the ALIGN program when comparing amino acid sequences. The BLAST programs of Altschul, et al., (1990) J. Mol. Biol. 215:403 are based on the algorithm of Karlin and Altschul, (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul, et al., (1997) Nucleic Acids Res. 25:3389. Alternatively, PSI-BLAST (in BLAST 2.0) can be used to perform an iterated search that detects distant relationships between molecules. See, Altschul, et al., (1997) supra. When utilizing BLAST, Gapped BLAST, PSI-BLAST, the default parameters of the respective programs (e.g., BLASTN for nucleotide sequences, BLASTX for proteins) can be used. See, http://www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2 and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch, (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG® Wisconsin Genetics Software Package® for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG® Wisconsin Genetics Software Package® is BLOSUM62 (see, Henikoff and Henikoff, (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, optimally at least 80%, more optimally at least 90% and most optimally at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more optimally at least 70%, 80%, 90% and most optimally at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, optimally 80%, more optimally 85%, most optimally at least 90% or 95% sequence identity to the reference sequence over a specified comparison window. Optimally, optimal alignment is conducted using the homology alignment algorithm of Needleman and Wunsch, (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

The invention further provides plants, plant cells and plant parts having altered levels and/or activities of an ACS3 polypeptide of the invention. In some embodiments, the plants of the invention have stably incorporated an ACS3 sequence of the invention. As discussed elsewhere herein, altering the level/activity of the ACS3 sequences of the invention can produce a variety of phenotypes.

### Methods

The use of the term “nucleotide construct” or “polynucleotide” herein is not intended to limit the present invention to nucleotide constructs comprising DNA. Those of ordinary skill in the art will recognize that nucleotide constructs, particularly polynucleotides and oligonucleotides, comprised of ribonucleotides and combinations of ribonucleotides and deoxyribonucleotides may also be employed in the methods disclosed herein. Thus, the nucleotide constructs of the present invention encompass all nucleotide constructs that can be employed in the methods of the present invention for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs of the invention also encompass all forms of nucleotide constructs including, but not limited to, single-stranded forms, double-stranded forms, hairpins, stem-and-loop structures and the like.

The nucleic acid sequences of the present invention can be introduced/expressed in a host cell such as bacteria, yeast, insect, mammalian or optimally plant cells. It is expected that those of skill in the art are knowledgeable in the numerous systems available for the introduction of a polypeptide or a nucleotide sequence of the present invention. No attempt to describe in detail the various methods known for providing proteins in prokaryotes or eukaryotes will be made.

The ACS3 sequences of the invention can be provided in expression cassettes for expression in the plant of interest. The cassette can include 5′ and 3′ regulatory sequences operably linked to an ACS3 sequence of the invention. “Operably linked” is intended to mean a functional linkage between two or more elements. For example, an operable linkage between a polynucleotide of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. An expression cassette may have a plurality of restriction sites for insertion of the ACS3 sequence to be under the transcriptional regulation of the regulatory regions. The expression cassette may additionally contain selectable marker genes.

The expression cassette can include in the 5′-3′ direction of transcription, a transcriptional initiation region (i.e., a promoter) and translational initiation region, an ACS3 sequence of the invention and a transcriptional and translational termination region (i.e., termination region) functional in plants. The promoter may be native/analogous or foreign to the plant host and/or to the ACS3 sequence of the invention. Additionally, the promoter may be a natural sequence or alternatively a synthetic sequence. Where the promoter is “foreign” to the plant host, it is intended that the promoter is not found in the native plant into which the promoter is introduced. Where the promoter is “foreign” to the ACS3 sequence of the invention, it is intended that the promoter is not the native or naturally occurring promoter for the operably linked ACS3 sequence of the invention. As used herein, a chimeric gene comprises a coding sequence operably linked to a transcription initiation region that is heterologous to the coding sequence.

While it may be optimal to express the sequences using foreign promoters, the native promoter sequences may be used. Such constructs would change expression levels of ACS3 in the plant or plant cell. Thus, the phenotype of the plant or plant cell can be altered.

The termination region may be native with the transcriptional initiation region, may be native with the operably linked ACS3 sequence of interest, may be native with the plant host or may be derived from another source (i.e., foreign to the promoter, the ACS3 sequence of interest, the plant host or any combination thereof). Convenient termination regions are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acid Res. 15:9627-9639.

Where appropriate, the gene(s) may be optimized for increased expression in the transformed plant. That is, the genes can be synthesized using plant-preferred codons for improved expression. See, for example, Campbell and Gowri, (1990) Plant Physiol. 92:1-11 for a discussion of host-preferred codon usage. Methods are available in the art for synthesizing plant-preferred genes and species-preferred genes. See, for example, U.S. Pat. Nos. 5,380,831 and 5,436,391 and Murray, et al., (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats and other such well-characterized sequences that may be deleterious to gene expression. The G-C content of the sequence may be adjusted to levels average for a given cellular host, as calculated by reference to known genes expressed in the host cell. The sequence may be modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein, et al., (1989) Proc. Natl. Acad. Sci. USA 86:6126-6130); potyvirus leaders, for example, TEV leader (Tobacco Etch Virus) (Gallie, et al., (1995) Gene 165(2):233-238), MDMV leader (Maize Dwarf Mosaic Virus) and human immunoglobulin heavy-chain binding protein (BiP) (Macejak, et al., (1991) Nature 353:90-94); untranslated leader from the coat protein mRNA of alfalfa mosaic virus (AMV RNA 4) (Jobling, et al., (1987) Nature 325:622-625); tobacco mosaic virus leader (TMV) (Gallie, et al., (1989) in Molecular Biology of RNA, ed. Cech (Liss, New York), pp. 237-256) and maize chlorotic mottle virus leader (MCMV) (Lommel, et al., (1991) Virology 81:382-385). See also, Della-Cioppa, et al., (1987) Plant Physiol. 84:965-968.

In preparing the expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). See generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Aci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Baim, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin); Gill, et al., (1988) Nature 334:721-724. Such disclosures are herein incorporated by reference. The above list of selectable marker genes is not meant to be limiting. Any selectable marker gene can be used in the present invention.

A number of promoters can be used in the practice of the invention. The promoters can be selected based on the desired outcome. That is, the nucleic acid can be combined with constitutive, tissue-preferred, developmentally regulated or other promoters for expression in plants. Constitutive promoters include, for example, the core promoter of the Rsyn7 promoter and other constitutive promoters disclosed in PCT Application Publication Number 99/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026) and the like. Other constitutive promoters include, for example, U.S. Pat. Nos. 5,608,149; 5,608,144; 5,604,121; 5,569,597; 5,466,785; 5,399,680; 5,268,463; 5,608,142 and 6,177,611.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides and the tobacco PR-1a promoter, which is activated by salicylic acid. Other chemical-regulated promoters of interest include steroid-responsive promoters (see, for example, the glucocorticoid-inducible promoter in Schena, et al., (1991) Proc. Natl. Acad. Sci. USA 88:10421-10425 and McNellis, et al., (1998) Plant J. 14(2):247-257) and tetracycline-inducible and tetracycline-repressible promoters (see, for example, Gatz, et al., (1991) Mol. Gen. Genet. 227:229-237 and U.S. Pat. Nos. 5,814,618 and 5,789,156), herein incorporated by reference.

Tissue-preferred promoters can be utilized to target enhanced ZmACS3 expression within a particular plant tissue. Tissue-preferred promoters include Yamamoto, et al., (1997) Plant J. 12(2):255-265; Kawamata, et al., (1997) Plant Cell Physiol. 38(7):792-803; Hansen, et al., (1997) Mol. Gen. Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam, (1994) Results Probl. Cell Differ. 20:181-196; Orozco, et al., (1993) Plant Mol. Biol. 23(6):1129-1138; Matsuoka, et al., (1993) Proc Natl. Acad. Sci. USA 90(20):9586-9590 and Guevara-Garcia, et al., (1993) Plant J. 4(3):495-505. Such promoters can be modified, if necessary, for weak expression.

“Seed-preferred” promoters include both those promoters active during seed initiation and/or development, such as promoters of seed storage proteins, as well as those promoters active during seed germination. See, Thompson, et al., (1989) BioEssays 10:108, herein incorporated by reference. Such seed-preferred promoters include, but are not limited to, Cim1 (cytokinin-induced message); cZ19B1 (maize 19 kDa zein); and, milps (myo-inositol-1-phosphate synthase); (see, PCT Application Publication Number WO 00/11177 and U.S. Pat. No. 6,225,529, herein incorporated by reference). Gamma-zein is another endosperm-specific promoter (Boronat, et al., (1986) Plant Science 47:95-102). Globulin-1 (Glob-1) is a preferred embryo-specific promoter. For dicots, seed-specific promoters include, but are not limited to, bean β-phaseolin, napin, β-conglycinin, soybean lectin, cruciferin and the like. For monocots, seed-preferred promoters include, but are not limited to, maize 15 kDa, 22 kDa zein, 27 kDa zein, gamma-zein, waxy, shrunken 1, shrunken 2, globulin 1, etc. See also, PCT Application Publication Number WO 00/12733, where seed-preferred promoters from end1 and end2 genes are disclosed, herein incorporated by reference. Additional seed-preferred promoters include the oleosin promoter (PCT Application Publication Number WO 00/0028058), the lipid transfer protein (LTP) promoter (U.S. Pat. No. 5,525,716). Additional seed-preferred promoters include the Lec1 promoter, the Jip1 promoter and the milps3 promoter (see, PCT Application Publication Number WO 02/42424).

The methods of the invention involve introducing a nucleotide construct or a polypeptide into a plant. By “introducing” is intended presenting to the plant the nucleotide construct (i.e., DNA or RNA) or polypeptide in such a manner that the nucleic acid or the polypeptide gains access to the interior of a cell of the plant. The methods of the invention do not depend on a particular method for introducing the nucleotide construct or the polypeptide to a plant, only that the nucleotide construct or polypeptide gains access to the interior of at least one cell of the plant. Methods for introducing nucleotide constructs and/or polypeptide into plants are known in the art including, but not limited to, stable transformation methods, transient transformation methods and virus-mediated methods.

By “stable transformation” is intended that the nucleotide construct introduced into a plant integrates into the genome of the plant and is capable of being inherited by progeny thereof. By “transient transformation” is intended that a nucleotide construct or the polypeptide introduced into a plant does not integrate into the genome of the plant.

Thus the ACS3 sequences of the invention can be provided to a plant using a variety of transient transformation methods including, but not limited to, the introduction of ACS3 protein or variants thereof directly into the plant and the introduction of the an ACS3 transcript into the plant. Such methods include, for example, microinjection or particle bombardment. See, for example, Crossway, et al., (1986) Mol. Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784, all of which are herein incorporated by reference. Alternatively, the various viral vector systems can be used for transient expression or the ACS3 nucleotide construct can be precipitated in a manner that precludes subsequent release of the DNA (thus, transcription from the particle-bound DNA can occur, but the frequency with which it is released to become integrated into the genome is greatly reduced).

The nucleotide constructs of the invention may be introduced into plants by contacting plants with a virus or viral nucleic acids. Generally, such methods involve incorporating a nucleotide construct of the invention within a viral DNA or RNA molecule. It is recognized that the an ACS3 polypeptide of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing nucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931, herein incorporated by reference.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (Townsend, et al., U.S. Pat. No. 5,563,055; Zhao, et al., U.S. Pat. No. 5,981,840), direct gene transfer (Paszkowski, et al., (1984) EMBO J. 3:2717-2722), and ballistic particle acceleration (see, for example, Sanford, et al., U.S. Pat. No. 4,945,050; Tomes, et al., U.S. Pat. No. 5,879,918; Tomes, et al., U.S. Pat. No. 5,886,244; Bidney, et al., U.S. Pat. No. 5,932,782; Tomes, et al., (1995) “Direct DNA Transfer into Intact Plant Cells via Microprojectile Bombardment,” in Plant Cell, Tissue, and Organ Culture: Fundamental Methods, ed. Gamborg and Phillips, (Springer-Verlag, Berlin); McCabe, et al., (1988) Biotechnology 6:923-926) and Lec1 transformation (PCT Application Publication Number WO 00/28058). Also see, Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); Tomes, U.S. Pat. No. 5,240,855; Buising, et al., U.S. Pat. Nos. 5,322,783 and 5,324,646; Tomes, et al., (1995) “Direct DNA Transfer into Intact Plant Cells via Microprojectile Bombardment,” in Plant Cell, Tissue, and Organ Culture: Fundamental Methods, ed. Gamborg, (Springer-Verlag, Berlin) (maize); Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; Bowen, et al., U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens), all of which are herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, PCT Application Publication Numbers WO 99/25821, WO 99/25854, WO 99/25840, WO 99/25855 and WO 99/25853, all of which are herein incorporated by reference. Briefly, the polynucleotide of the invention can be contained in a transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick, et al., (1986) Plant Cell Reports 5:81-84. These plants may then be grown and either pollinated with the same transformed strain or different strains and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis) and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima) and chrysanthemum.

Conifers that may be employed in practicing the present invention include, for example, pines such as loblolly pine (Pinus taeda), slash pine (Pinus elliotii), ponderosa pine (Pinus ponderosa), lodgepole pine (Pinus contorta) and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea) and cedars such as Western red cedar (Thuja plicata) and Alaska yellow-cedar (Chamaecyparis nootkatensis). Optimally, plants of the present invention are crop plants (for example, corn, alfalfa, sunflower, Brassica, soybean, cotton, safflower, peanut, sorghum, wheat, millet, tobacco, etc.), more optimally corn and soybean plants, yet more optimally corn plants.

Plants of particular interest include grain plants that provide seeds of interest, oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Typically, an intermediate host cell will be used in the practice of this invention to increase the copy number of the cloning vector. With an increased copy number, the vector containing the nucleic acid of interest can be isolated in significant quantities for introduction into the desired plant cells. In one embodiment, plant promoters that do not cause expression of the polypeptide in bacteria are employed.

Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli. is also useful. Examples of such markers include genes specifying resistance to ampicillin, tetracycline or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., (1983) Gene 22:229-235); Mosbach, et al., (1983) Nature 302:543-545).

A variety of eukaryotic expression systems such as yeast, insect cell lines, plant and mammalian cells, are known to those of skill in the art. As explained briefly below, a polynucleotide of the present invention can be expressed in these eukaryotic systems. In some embodiments, transformed/transfected plant cells, as discussed infra, are employed as expression systems for production of the proteins of the instant invention.

Synthesis of heterologous polynucleotides in yeast is well known (Sherman, et al., (1982) Methods in Yeast Genetics, Cold Spring Harbor Laboratory). Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein of the present invention, once expressed, can be isolated from yeast by lysing the cells and applying standard protein isolation techniques to the lists. The monitoring of the purification process can be accomplished by using Western blot techniques or radioimmunoassay of other standard immunoassay techniques.

The sequences of the present invention can also be ligated to various expression vectors for use in transfecting cell cultures of, for instance, mammalian, insect or plant origin. Illustrative cell cultures useful for the production of the peptides are mammalian cells. A number of suitable host cell lines capable of expressing intact proteins have been developed in the art, and include the HEK293, BHK21 and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site) and transcriptional terminator sequences. Other animal cells useful for production of proteins of the present invention are available, for instance, from the American Type Culture Collection.

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See, Schneider, (1987) J. Embryol. Exp. Morphol. 27:353-365).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, et al., (1983) J. Virol. 45:773-781). Additionally, gene sequences to control replication in the host cell may be incorporated into the vector such as those found in bovine papilloma virus type-vectors (Saveria-Campo, (1985) DNA Cloning Vol. II a Practical Approach, Glover, Ed., IRL Press, Arlington, Va., pp. 213-238).

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics and micro-injection of the DNA directly into the cells. The transfected cells are cultured by means well known in the art (Kuchler, (1997) Biochemical Methods in Cell Culture and Virology, Dowden, Hutchinson and Ross, Inc.).

In some embodiments, the content and/or composition of polypeptides of the present invention in a plant may be modulated by altering, in vivo or in vitro, the promoter of a gene to up- or down-regulate gene expression. In some embodiments, the coding regions of native genes of the present invention can be altered via substitution, addition, insertion or deletion to decrease activity of the encoded enzyme. See, e.g., Kmiec, U.S. Pat. No. 5,565,350; Zarling, et al., PCT/US93/03868. In other embodiments, the polypeptide of the invention is introduced. And in some embodiments, an isolated nucleic acid (e.g., a vector) comprising a promoter sequence is transfected into a plant cell. Subsequently, a plant cell comprising the promoter operably linked to a polynucleotide of the present invention is selected for by means known to those of skill in the art such as, but not limited to, Southern blot, DNA sequencing or PCR analysis using primers specific to the promoter and to the gene and detecting amplicons produced therefrom. A plant or plant part altered or modified by the foregoing embodiments is grown under plant forming conditions for a time sufficient to modulate the concentration and/or composition of polypeptides of the present invention in the plant. Plant forming conditions are well known in the art and discussed briefly, supra.

A method for modulating the concentration and/or activity of the polypeptide of the present invention is provided. By “modulation” is intended any alteration in the level and/or activity (i.e., increase or decrease) that is statistically significant compared to a control plant or plant part. In general, concentration, composition or activity is increased or decreased by at least 5%, 10%, 20%, 30%, 40%, 50%, 60%, 70%, 80% or 90% relative to a control plant, plant part or cell. The modulation may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of inducing compound. Inducible promoters and inducing compounds, which activate expression from these promoters, are well known in the art. In specific embodiments, the polypeptides of the present invention are modulated in monocots, particularly maize.

The level of the ACS3 polypeptide, and/or the effect of modulating ACS3 expression, may be measured directly, for example, by assaying the level of the ACS3 RNA or polypeptide in the plant; or indirectly, for example, by measuring the ACS3 activity of the ACS3 polypeptide in the plant, or by measuring the level or activity of ACC. Methods for determining ACS3 level or activity are described elsewhere herein or known in the art. ACC may be assayed by gas chromatography-mass spectroscopy; see also, Methods in Plant Biochemistry and Molecular Biology (1997) CRC Press, Ed. W. Dashek, at Chapter 12, pp. 158-159. Further, modified plants may be assayed for ethylene production. The concentration of ethylene can be determined by, e.g., gas chromatography-mass spectroscopy, etc. See, e.g., Nagahama, et al., (1991) J. Gen. Microbiol. 137:2281-2286. For example, ethylene can be measured with a gas chromatograph equipped with, e.g., an alumina based column (such as an HP-PLOT A1203 capillary column (Agilent Technologies, Santa Clara, Calif.)) and a flame ionization detector.

Phenotypic analysis includes, e.g., analyzing changes in chemical composition, morphology or physiological properties of the plant. For example, phenotypic changes associated with modulation of ACS3 expression can include, but are not limited to, an increase in drought tolerance, an increase in density tolerance, an increase in nitrogen use efficiency, an increase in yield under optimal conditions, and/or a decrease in ethylene production.

A variety of assays can be used for monitoring drought tolerance and/or NUE. For example, assays include, but are not limited to, visual inspection, monitoring photosynthesis measurements and measuring levels of chlorophyll, DNA, RNA and/or protein content of, e.g., the leaves, under stress and non-stress conditions.

In specific embodiments, the polypeptide or the polynucleotide of the invention is introduced into the plant cell. Subsequently, a plant cell having the introduced sequence of the invention is selected using methods known to those of skill in the art such as, but not limited to, Southern blot analysis, DNA sequencing, PCR analysis or phenotypic analysis. A plant or plant part altered or modified by the foregoing embodiments is grown under plant forming conditions for a time sufficient to modulate the concentration and/or activity of polypeptides of the present invention in the plant. Plant forming conditions are well known in the art and discussed briefly elsewhere herein.

It is also recognized that the level and/or activity of the polypeptide may be modulated by employing a polynucleotide that is not capable of directing, in a transformed plant, the expression of a protein or an RNA. For example, the polynucleotides of the invention may be used to design polynucleotide constructs that can be employed in methods for altering or mutating a genomic nucleotide sequence, or its expression, in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides and recombinogenic oligonucleobases. Such nucleotide constructs and methods of use are known in the art. See, U.S. Pat. Nos. 5,565,350; 5,731,181; 5,756,325; 5,760,012; 5,795,972 and 5,871,984, all of which are herein incorporated by reference. See also, PCT Application Publication Number WO 98/49350, WO 99/07865, WO 99/25821 and Beetham, et al., (1999) Proc. Natl. Acad. Sci. USA 96:8774-8778, herein incorporated by reference.

It is therefore recognized that methods of the present invention do not depend on the incorporation of the entire polynucleotide into the genome, only that the plant or cell thereof is altered as a result of the introduction of the polynucleotide into a cell. In one embodiment of the invention, the genome may be altered following the introduction of the polynucleotide into a cell. For example, the polynucleotide, or any part thereof, may incorporate into the genome of the plant. Alterations to the genome of the present invention include, but are not limited to, additions, deletions and substitutions of nucleotides into the genome. While the methods of the present invention do not depend on additions, deletions and substitutions of any particular number of nucleotides, it is recognized that such additions, deletions or substitutions comprise at least one nucleotide.

In some embodiments, the activity and/or level of the ACS3 polypeptide of the invention is increased. An increase in the level or activity of the ACS3 polypeptide of the invention can be achieved by providing to the plant an ACS3 polypeptide. As discussed elsewhere herein, many methods are known the art for providing a polypeptide to a plant including, but not limited to, direct introduction of the polypeptide into the plant and/or introducing into the plant (transiently or stably) a nucleotide construct encoding a polypeptide having ACS3 activity. In other embodiments, the level or activity of an ACS3 polypeptide may be increased by altering the gene encoding the ACS3 polypeptide or its promoter. See, e.g., U.S. Pat. No. 5,565,350 and PCT/US93/03868. The invention therefore encompasses mutagenized plants that carry mutations in ACS3 genes, where the mutations increase expression of the ACS3 gene or increase the ACS3 activity of the encoded ACS3 polypeptide.

In some embodiments, the activity and/or level of the ACS3 polypeptide of the invention of is reduced or eliminated by introducing into a plant a polynucleotide that inhibits the level or activity of the ACS3 polypeptide of the invention. The introduced polynucleotide may inhibit the expression of ACS3 directly, by preventing translation of the ACS3 messenger RNA, or indirectly, by encoding a polypeptide that inhibits the transcription or translation of an ACS3 gene encoding an ACS3 protein. Methods for inhibiting or eliminating the expression of a gene in a plant are well known in the art and any such method may be used in the present invention to inhibit the expression of ACS3 in a plant. In other embodiments of the invention, the activity of ACS3 polypeptide is reduced or eliminated by transforming a plant cell with an expression cassette comprising a polynucleotide encoding a polypeptide that inhibits the activity of the ACS3 polypeptide. In certain embodiments, the activity of an ACS3 polypeptide may be reduced or eliminated by disrupting the gene encoding the ACS3 polypeptide. The invention encompasses mutagenized plants that carry mutations in ACS3 genes, where the mutations reduce expression of the ACS3 gene or inhibit the ACS3 activity of the encoded ACS3 polypeptide.

Reduction of the activity of specific genes (also known as gene silencing or gene suppression) is desirable for several aspects of genetic engineering in plants. Methods for inhibiting gene expression are well known in the art and include, but are not limited to, homology-dependent gene silencing, antisense technology, RNA interference (RNAi) and the like. The general term, homology-dependent gene silencing, encompasses the phenomenon of cis-inactivation, trans-inactivation and cosuppression. See, Finnegan, et al., (1994) Biotech. 12:883-888 and Matzke, et al., (1995) Plant Physiol. 107:679-685, both incorporated herein in their entirety by reference. These mechanisms represent cases of gene silencing that involve transgene/transgene or transgene/endogenous gene interactions that lead to reduced expression of protein in plants. A “transgene” is a recombinant DNA construct that has been introduced into the genome by a transformation procedure. As one alternative, incorporation of antisense RNA into plants can be used to inhibit the expression of endogenous genes and produce a functional mutation within the genome. The effect is achieved by introducing into the cell(s) DNA that encodes RNA that is complementary to the sequence of mRNA of the target gene. See, e.g., Bird, et al., (1991) Biotech and Gen. Eng. Rev. 9:207-226, incorporated herein in its entirety by reference. See also, the more detailed discussion herein below addressing these and other methodologies for achieving inhibition of expression or function of a gene.

Many techniques for gene silencing are well known to one of skill in the art, including, but not limited to, antisense technology (see, e.g., Sheehy, et al., (1988) Proc. Natl. Acad. Sci. USA 85:8805-8809 and U.S. Pat. Nos. 5,107,065; 5,453,566 and 5,759,829); cosuppression (e.g., Taylor, (1997) Plant Cell 9:1245; Jorgensen, (1990) Trends Biotech. 8(12):340-344; Flavell, (1994) Proc. Natl. Acad. Sci. USA 91:3490-3496; Finnegan, et al., (1994) Bio/Technology 12:883-888 and Neuhuber, et al., (1994) Mol. Gen. Genet. 244:230-241); RNA interference (Napoli, et al., (1990) Plant Cell 2:279-289; U.S. Pat. No. 5,034,323; Sharp, (1999) Genes Dev. 13:139-141; Zamore, et al., (2000) Cell 101:25-33 and Montgomery, et al., (1998) Proc. Natl. Acad. Sci. USA 95:15502-15507), virus-induced gene silencing (Burton, et al., (2000) Plant Cell 12:691-705 and Baulcombe, (1999) Curr. Op. Plant Bio. 2:109-113); target-RNA-specific ribozymes (Haseloff, et al., (1988) Nature 334:585-591); hairpin structures (Smith, et al., (2000) Nature 407:319-320; PCT Application Publication Numbers WO 99/53050; WO 02/00904; WO 98/53083; Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7, US Patent Application Publication Number 2003/0175965; Panstruga, et al., (2003) Mol. Biol. Rep. 30:135-140; Wesley, et al., (2001) Plant J. 27:581-590; Wang and Waterhouse, (2001) Curr. Opin. Plant Biol. 5:146-150; US Patent Application Publication Number 2003/0180945 and PCT Application Publication Number WO 02/00904, all of which are herein incorporated by reference); ribozymes (Steinecke, et al., (1992) EMBO J. 11:1525 and Perriman, et al., (1993) Antisense Res. Dev. 3:253); oligonucleotide-mediated targeted modification (e.g., PCT Application Publication Numbers WO 03/076574 and WO 99/25853); Zn-finger targeted molecules (e.g., PCT Application Publication Numbers WO 01/52620; WO 03/048345 and WO 00/42219); transposon tagging (Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928; Bensen, et al., (1995) Plant Cell 7:75-84; Mena, et al., (1996) Science 274:1537-1540 and U.S. Pat. No. 5,962,764), each of which is herein incorporated by reference and other methods or combinations of the above methods known to those of skill in the art.

It is recognized that with the polynucleotides of the invention, antisense constructions, complementary to at least a portion of the messenger RNA (mRNA) for the ACS3 sequences, can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, optimally 80%, more optimally 85% sequence identity to the corresponding antisensed sequences may be used. Furthermore, portions of the antisense nucleotides may be used to disrupt the expression of the target gene. Generally, sequences of at least 15 nucleotides, 20 nucleotides, 25 nucleotides, 30 nucleotides, 35 nucleotides, 40 nucleotides, 44 nucleotides, 50 nucleotides, 100 nucleotides, 200 nucleotides or 300, 400, 450, 500, 550 or more nucleotides may be used.

The polynucleotides of the present invention may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a polynucleotide that corresponds to the transcript of the endogenous gene. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323, herein incorporated by reference. Thus, many methods may be used to reduce or eliminate the activity of an ACS polypeptide. More than one method may be used to reduce the activity of a single ACS polypeptide. In addition, combinations of methods may be employed to reduce or eliminate the activity of multiple ACS polypeptides.

Furthermore, it is recognized that the methods of the invention may employ a nucleotide construct that is capable of directing, in a transformed plant, the expression of at least one protein or at least one RNA, such as, for example, an antisense RNA that is complementary to at least a portion of an mRNA. Typically such a nucleotide construct is comprised of a coding sequence for a protein or an RNA operably linked to 5′ and 3′ transcriptional regulatory regions. Alternatively, it is also recognized that the methods of the invention may employ a nucleotide construct that is not capable of directing, in a transformed plant, the expression of a protein or an RNA.

Modulation of the ACS3 polynucleotides of the present invention can also be combined with modulation of other genes implicated in regulation of, production of or response to ethylene. The combinations generated can also include multiple copies of any one of the polynucleotides of interest. The combinations may have any combination of up-regulating and down-regulating expression of the combined polynucleotides. The combinations may or may not be combined on one construct for transformation of the host cell, and therefore may be provided sequentially or simultaneously. The host cell may be a wild-type or mutant cell, in a normal or aneuploid state.

Methods to assay for an increase in seed set during abiotic stress are known in the art. For example, plants comprising ACS3 sequences of the invention can be monitored under various stress conditions and compared to control plants. For instance, a plant having an ACS3 downregulation construct can be subjected to various degrees of stress during flowering and seed set. Under comparable conditions, the genetically modified plant comprising the ACS3 downregulation construct will have a higher number of developing seeds than a wild type (non-transformed) plant.

Accordingly, the present invention further provides plants having increased yield or maintaining their yield during periods of abiotic stress (i.e. drought, salt, heavy metals, temperature, etc). In some embodiments, the plants having an increased or maintained yield during abiotic stress have an increased level/activity of the ACS3 polypeptide of the invention. In certain embodiments, the plant comprises a heterologous ACS3 nucleotide sequence of the invention operably linked to a promoter that drives expression in the plant cell. In certain embodiments, such plants have stably incorporated into their genome a heterologous nucleic acid molecule comprising an ACS3 nucleotide sequence of the invention operably linked to a promoter that drives expression in the plant cell. The ACS3 nucleotide sequence may be in a construct designed for downregulation of expression of ACS3, as described elsewhere herein.

In another embodiment, a method of transforming in a plant is provided. The method comprises providing a target plant, where the target plant had been provided an ACS3 sequence of the invention. In some embodiments, the ACS3 nucleotide sequence is provided by introducing into the plant a heterologous polynucleotide comprising an ACS3 nucleotide sequence of the invention, expressing the ACS3 sequence. In yet other embodiments, the ACS3 nucleotide construct introduced into the target plant is stably incorporated into the genome of the plant. The target plant is transformed with a polynucleotide of interest. It is recognized that the target plant having had the ACS3 sequence introduced (referred to herein as a “modified target plant”), can be grown under conditions to produce at least one cell division to produce a progeny cell expressing the ACS3 sequence prior to transformation with one or more polynucleotides of interest. As used herein “re-transformation” refers to the transformation of a modified cell.

The modified target cells having been provided the ACS3 sequence can be obtained from T0 transgenic cultures, regenerated plants or progeny whether grown in vivo or in vitro so long as they exhibit stimulated growth compared to a corresponding cell that does not contain the modification. This includes but is not limited to transformed callus, tissue culture, regenerated T0 plants or plant parts such as immature embryos or any subsequent progeny of T0 regenerated plants or plant parts.

Once the target cell is provided with the ACS3 nucleotide sequence it may be re-transformed with at least one gene of interest. The transformed cell can be from transformed callus, transformed embryo, T0 regenerated plants or its parts, progeny of T0 plants or parts thereof as long as the ACS3 sequence of the invention is stably incorporated into the genome.

Methods to determine transformation efficiencies or the successful transformation of the polynucleotide of interest are known in the art. For example, transgenic plants expressing a selectable marker can be screened for transmission of the gene(s) of interest using, for example, chemical selection, phenotype screening standard immunoblot and DNA detection techniques. Transgenic lines are also typically evaluated on levels of expression of the heterologous nucleic acid. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes.

The RNA-positive plants can then be analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

Seeds derived from plants regenerated from re-transformed plant cells, plant parts or plant tissues or progeny derived from the regenerated plants, may be used directly as feed or food or further processing may occur.

Any polynucleotide of interest can be used in the methods of the invention, for example in combination with ACS3 modification. Various changes in phenotype are of interest, including modifying the fatty acid composition in a plant; altering the amino acid content, starch content or carbohydrate content of a plant; altering a plant's pathogen defense mechanism; affecting kernel size or sucrose loading, and the like. The gene of interest may also be involved in regulating the influx of nutrients and in regulating expression of phytate genes, particularly to reduce phytate levels in the seed. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate or nutrient metabolism as well as those affecting kernel size, sucrose loading and the like.

Agronomically important traits such as oil, starch and protein content can be genetically altered in addition to using traditional breeding methods. Modifications include increasing content of oleic acid, saturated and unsaturated oils, increasing levels of lysine and sulfur, providing essential amino acids and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016 and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502, herein incorporated by reference); corn (Pedersen, et al., (1986) J. Biol. Chem. 261:6279; Kirihara, et al., (1988) Gene 71:359, both of which are herein incorporated by reference) and rice (Musumura, et al., (1989) Plant Mol. Biol. 12:123, herein incorporated by reference) could be used. Other agronomically important genes encode latex, Floury 2, growth factors, seed storage factors and transcription factors.

Insect resistance genes may encode resistance to pests that have great yield drag such as rootworm, cutworm, European Corn Borer and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109) and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432 and Mindrinos, et al., (1994) Cell 78:1089) and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), glyphosate (e.g., the EPSPS gene and the GAT gene; see, for example, US Patent Application Publication Number 2004/0082770 and PCT Application Publication WO 03/092360) or other such genes known in the art. The bar gene encodes resistance to the herbicide basta, the nptII gene encodes resistance to the antibiotics kanamycin and geneticin and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids and levels of cellulose. In corn, modified hordothionin proteins are described in U.S. Pat. Nos. 5,703,049, 5,885,801, 5,885,802 and 5,990,389.

Commercial traits can also be encoded on a gene or genes that could increase for example, starch for ethanol production or provide expression of proteins. Another important commercial use of transformed plants is the production of polymers and bioplastics such as described in U.S. Pat. No. 5,602,321. Genes such as 13-Ketothiolase, PHBase (polyhydroxyburyrate synthase) and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones and the like. The level of proteins, particularly modified proteins having improved amino acid distribution to improve the nutrient value of the plant, can be increased. This is achieved by the expression of such proteins having enhanced amino acid content.

The following examples are offered by way of illustration and not by way of limitation.

### EXPERIMENTAL

### Example 1

### Variants of Zm-ACS3

A. Variant Nucleotide Sequences of Zm-ACS3 (SEQ ID NO: 1) that do not Alter the Encoded Amino Acid Sequence

The Zm-ACS3 nucleotide sequence set forth in SEQ ID NO: 1 can be used to generate variant nucleotide sequences having the nucleotide sequence of the open reading frame with about 70%, 75%, 80%, 85%, 90%, 95%, 96%, 97%, 98%, or 99% nucleotide sequence identity when compared to the starting unaltered open reading frame nucleotide sequence of SEQ ID NO: 1 (genomic) or SEQ ID NO: 2 (coding sequence). These functional variants are generated using a standard codon table. While the nucleotide sequence of the variant is altered, the amino acid sequence encoded by the open reading frame does not change.

B. Variant Amino Acid Sequences of Zm-ACS3

Variant amino acid sequences of Zm-ACS3 can be generated. Specifically, the open reading frame set forth in SEQ ID NO: 2 is reviewed to determine appropriate amino acid alteration. The selection of the amino acid to change is made by aligning the protein sequence with orthologs and other gene family members from various species. An amino acid is selected that is deemed not to be under high selection pressure (not highly conserved) and which could be rather easily substituted by an amino acid with similar chemical characteristics (i.e., similar functional side-chain). Additional alterations can be made following the same steps and with the judicious application of an amino acid substitutions table, such as Table 2.

TABLE 2
Substitution Table

| Amino | Strongly Similar and | Rank of Order | |
|---|---|---|---|
| Acid | Optimal Substitution | to Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine |
| | | | cannot change |
| H | | Na | No good substitutes |
| C | | Na | No good substitutes |
| P | | Na | No good substitutes |

First, any conserved amino acids in the protein that should not be changed are identified and marked for insulation from substitution. The start methionine will of course be added to this list automatically. Next, the changes are made.

H, C and P will not be changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, starting with as many isoleucine changes as needed before leucine, and so on down to methionine. Clearly many amino acids will in this manner not need to be changed. L, I and V will involve a 50:50 substitution of the two alternate optimal substitutions.

### Example 2

### Agrobacterium-Mediated Transformation

For Agrobacterium-mediated transformation of maize with a plasmid containing the Zm-ACS3 sequence operably linked to a plant promoter and the selectable marker gene PAT, optimally the method of Zhao is employed (U.S. Pat. No. 5,981,840 and PCT Application Publication Number WO 98/32326, the contents of which are hereby incorporated by reference). Briefly, immature embryos are isolated from maize and the embryos contacted with a suspension of Agrobacterium, where the bacteria are capable of transferring the ZmACS3 sequence to at least one cell of at least one of the immature embryos (step 1: the infection step). In this step the immature embryos are optimally immersed in an Agrobacterium suspension for the initiation of inoculation. The embryos are co-cultured for a time with the Agrobacterium (step 2: the co-cultivation step). Optimally the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Optimally the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Optimally, the immature embryos are cultured on solid medium with a selective agent resulting in the selective growth of transformed cells. The callus is then regenerated into plants (step 5: the regeneration step) and optimally calli grown on selective medium are cultured on solid medium to regenerate the plants.

### Example 3

### Biolistic Transformation and Regeneration of Maize Embryos

Immature maize embryos from greenhouse donor plants are bombarded with a plasmid containing an ACS3 sequence of the invention operably linked to a promoter. This could be a weak promoter such as nos, a tissue-specific promoter, such as globulin-1, an inducible promoter such as In2 or a strong promoter such as ubiquitin, plus a plasmid containing the selectable marker gene PAT (Wohlleben, et al., (1988) Gene 70:25-37) that confers resistance to the herbicide Bialaphos. Transformation is performed as follows.

Maize ears are harvested 8-14 days after pollination and surface sterilized in 30% Clorox® bleach plus 0.5% Micro detergent for 20 minutes and rinsed two times with sterile water. The immature embryos are excised and placed embryo axis side down (scutellum side up), 25 embryos per plate. These are cultured on 560L medium 4 days prior to bombardment in the dark. Medium 560L is an N6-based medium containing Eriksson's vitamins, thiamine, sucrose, 2,4-D and silver nitrate. The day of bombardment, the embryos are transferred to 560Y medium for 4 hours and are arranged within the 2.5-cm target zone. Medium 560Y is a high osmoticum medium (560L with high sucrose concentration).

A plasmid vector comprising the ACS3 sequence operably linked to the selected promoter is constructed. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water, 10 μl (1 μg) DNA in TrisEDTA buffer (1 μg total), 100 μl 2.5M CaC1², 10 μl 0.1M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 μl 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are positioned 2 levels below the stooping plate for bombardment in a DuPont Helium Particle Gun. All samples receive a single shot at 650 PSI, with a total of ten aliquots taken from each tube of prepared particles/DNA. As a control, embryos are bombarded with DNA containing the PAT selectable marker as described above but without the ACS3 sequence.

Following bombardment, the embryos are kept on 560Y medium, an N6 based medium, for 2 days, then transferred to 560R selection medium, an N6 based medium containing 3 mg/liter Bialaphos and subcultured every 2 weeks. After approximately 10 weeks of selection, bialaphos-resistant callus clones are sampled for PCR and activity of the gene of interest. Positive lines are transferred to 288J medium, an MS based medium with lower sucrose and hormone levels, to initiate plant regeneration. Following somatic embryo maturation (2-4 weeks), well-developed somatic embryos are transferred to medium for germination and transferred to the lighted culture room. Approximately 7-10 days later, developing plantlets are transferred to medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to Classic™ 600 pots (1.6 gallon) and grown to maturity. Plants are monitored for expression of the gene of interest.

### Example 4

### Soybean Embryo Transformation

Soybean embryos are bombarded with a plasmid containing the ACS3 sequence operably linked to a promoter. This could be a weak promoter such as nos, a tissue-specific promoter, such as globulin-1, an inducible promoter such as In2 or a strong promoter such as ubiquitin plus a plasmid containing the selectable marker gene PAT (Wohlleben, et al., (1988) Gene 70:25-37) that confers resistance to the herbicide Bialaphos. Transformation is performed as follows.

To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A DuPont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188) and the 3′ region of the nopaline synthase gene from the T-DNA of the Ti plasmid of Agrobacterium tumefaciens. The expression cassette comprising the ZmACS3 operably linked to the promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 5

### Sunflower Meristem Tissue Transformation Prophetic Example

Sunflower meristem tissues are transformed with an expression cassette containing the ACS3 sequence operably linked to a promoter. This could be a weak promoter such as nos, a tissue-specific promoter, such as globulin-1, an inducible promoter such as In2 or a strong promoter such as ubiquitin plus a plasmid containing the selectable marker gene PAT (Wohlleben, et al., (1988) Gene 70:25-37) that confers resistance to the herbicide Bialaphos. Transformation is performed as follows. See also, EP Patent Number EP 0 486233, herein incorporated by reference and Malone-Schoneberg, et al., (1994) Plant Science 103:199-207).

Mature sunflower seed (Helianthus annuus L.) are dehulled using a single wheat-head thresher. Seeds are surface sterilized for 30 minutes in a 20% Clorox® bleach solution with the addition of two drops of Tween® 20 per 50 ml of solution. The seeds are rinsed twice with sterile distilled water.

Split embryonic axis explants are prepared by a modification of procedures described by Schrammeijer, et al., (Schrammeijer, et al., (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al., (1962) Physiol. Plant., 15:473-497), Shepard's vitamin additions (Shepard, (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA₃), pH 5.6 and 8 g/l Phytagar®.

The explants may be subjected to microprojectile bombardment prior to Agrobacterium treatment (Bidney, et al., (1992) Plant Mol. Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000® particle acceleration device.

Disarmed Agrobacterium tumefaciens strain EHA105 may be used in transformation. A binary plasmid vector comprising the expression cassette that contains the ZmACS3 gene operably linked to the promoter is introduced into Agrobacterium strain EHA105 via freeze-thawing as described by Holsters, et al., (1978) Mol. Gen. Genet. 163:181-187. This plasmid further comprises a kanamycin selectable marker gene (i.e., nptII). Bacteria for plant transformation experiments are grown overnight (28° C. and 100 RPM continuous agitation) in liquid YEP medium (10 gm/l yeast extract, 10 gm/l Bacto®peptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for ZmACS3 activity.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% Gelrite®, pH 5.6) and grown under conditions described for explant culture. The upper portion of the seedling is removed, a 1 cm vertical slice is made in the hypocotyl, and the transformed shoot inserted into the cut. The entire area is wrapped with Parafilm® to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by ZmACS3 activity analysis of leaf extracts while transgenic seeds harvested from NPTII-positive T₀ plants are identified by ZmACS3 activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox® bleach solution with the addition of two to three drops of Tween® 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA and 0.8% Phytagar® at pH 5.6) for 24 hours under the dark. The primary leaves are removed to expose the apical meristem; approximately 40 explants are placed, with the apical dome facing upward, in a 2 cm circle in the center of 374M (GBA medium with 1.2% Phytagar®) and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bacto®peptone and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD₆₀₀. Particle-bombarded explants are transferred to GBA medium (374E) and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for ZmACS3 activity using assays known in the art. Explants positive for ZmACS3 presence and/or expression are identified and subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for ZmACS3 expression are grafted to Pioneer® hybrid 6440 in vitro-grown sunflower seedling rootstock. The rootstocks are prepared in the following manner. Seeds are dehulled and surface-sterilized for 20 minutes in a 20% Clorox® bleach solution with the addition of two to three drops of Tween® 20 per 100 ml of solution, and are rinsed three times with distilled water. The sterilized seeds are germinated on the filter moistened with water for three days, then they are transferred into 48 medium (half-strength MS salt, 0.5% sucrose, 0.3% Gelrite® pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm®. After one week of culture on the medium, grafted plants are transferred to soil. In the first two weeks, they are maintained under high humidity conditions to acclimatize to a greenhouse environment.

### Example 6

### Identification of Homologous Genes and Gene Family Members

cDNA clones encoding the polypeptide of interest can be identified by conducting BLAST (Basic Local Alignment Search Tool; Altschul, et al., (1993) J. Mol. Biol. 215:403-410; see also, the explanation of the BLAST algorithm on the world wide web site for the National Center for Biotechnology Information at the National Library of Medicine of the National Institutes of Health) searches for similarity to amino acid sequences contained in the BLAST “nr” database (comprising all non-redundant GenBank CDS translations, sequences derived from the 3 dimensional structure Brookhaven Protein Data Bank, the last major release of the SWISS PROT protein sequence database, EMBL and DDBJ databases). The DNA sequences from clones can be translated in all reading frames and compared for similarity to all publicly available protein sequences contained in the “nr” database using the BLASTX algorithm (Gish and States, (1993) Nat. Genet. 3:266-272) provided by the NCBI. The polypeptides encoded by the cDNA sequences can be analyzed for similarity to all publicly available amino acid sequences contained in the “nr” database using the BLASTP algorithm provided by the National Center for Biotechnology Information (NCBI). For convenience, the P value (probability) and the E-value (expectation) of observing a match of a cDNA-encoded sequence to a sequence contained in the searched databases merely by chance, as calculated by BLAST, are reported as “pLog” values, which represent the negative of the logarithm of the reported P value or E value. Accordingly, the greater the pLog value, the greater the likelihood that the cDNA-encoded sequence and the BLAST “hit” represent homologous proteins.

ESTs (expressed sequence tags) can be compared to the GenBank database as described above. ESTs that contain sequences more 5-prime or 3-prime can be found by using the BLASTN algorithm (Altschul, et al., (1997) Nucleic Acids Res. 25:3389-3402.) against the DUPONT™ proprietary database, comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5-prime or 3-prime direction. Once the most 5-prime EST is identified, its complete sequence can be determined by Full Insert Sequencing. Homologous genes from genomic sequence or belonging to different species can be found by comparing the amino acid sequence of a known gene (from either a proprietary source or a public database) against a genomic database or an EST database using the TBLASTN algorithm. The TBLASTN algorithm searches an amino acid query against a nucleotide database that is translated in all 6 reading frames. This search allows for differences in nucleotide codon usage between different species and for codon degeneracy.

Genomic sequence can be analyzed using the FGENESH (Salamov and Solovyev, (2000) Genome Res. 10:516-522) program, and optionally, can be aligned with homologous sequences from other species to assist in identification of putative introns. A genomic sequence can be pre-analyzed using FGENESH to identify putative coding sequences; these sequences can be translated and the known gene can be compared to these sequences using BLASTP.

Using such methods, a new maize ACS gene was identified from two contigs in a proprietary database, PCO527070 and PCO663271, and designated ZmACS3. The gene is located on the short arm of chromosome 3, at approximately 30.9 cM; the border markers are MZA8206 and MZA1301. It contains 3 exons and 2 introns and encodes a polypeptide of 462 amino acids.

### Example 7

### Transgenic Downregulation of ACS3 Expression

Down-regulation of ACC synthase(s), e.g., by hairpin RNA (hpRNA) expression, can result in plants or plant cells having reduced expression (up to and including no detectable expression) of one or more ACC synthases. Further, expression of hpRNA molecules specific for one or more ACC synthase genes (e.g., targeting one or more ACC synthase coding regions, promoters, or untranslated regions) in plants can alter phenotypes such as ethylene production, drought tolerance, density tolerance, seed or biomass yield and/or nitrogen use efficiency of the plants.

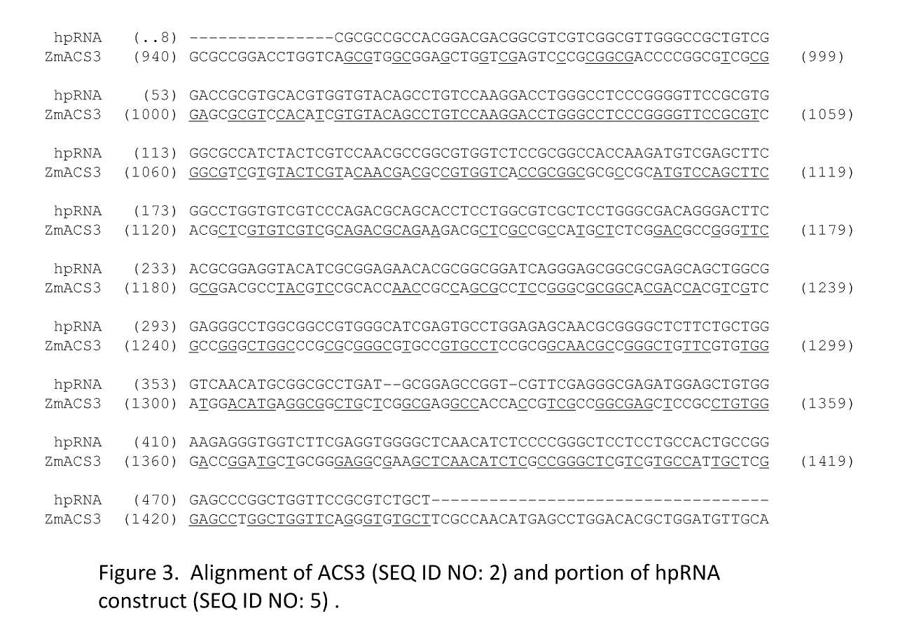

ZmACS3 expression was evaluated in maize plants heterozygous for a transgenic expression cassette comprising an ACC synthase polynucleotide sequence configured for RNA silencing or interference, as described more fully in U.S. patent application Ser. No. 12/897,489, filed Oct. 4, 2010; see SEQ ID NO: 7 and FIG. 4. This cassette comprises a 487 bp inverted repeat region from ZmACS6. Within the region are 44 contiguous base pairs that are 100% identical to a region of ZmACS3, as shown in FIG. 3, which may act to downregulate ZmACS3, a previously unrecognized gene in the ethylene biosynthesis pathway.

In order to evaluate event efficacy, plants representing ten independent transgenic events were analyzed. Eight-day-old maize seedlings (stage V3) were subjected to flooding for 30 hours. Root tissue was then collected from transgenic (TG) and corresponding nontransgenic (WT) seedlings, and ZmACS6 and ZmACS3 transcript levels were measured using standard qRT-PCR techniques. The ZmACS3 primers (SEQ ID NOs: 8 and 9) were designed to target the 3′ region of the second exon, generating an amplicon of 70 bp.

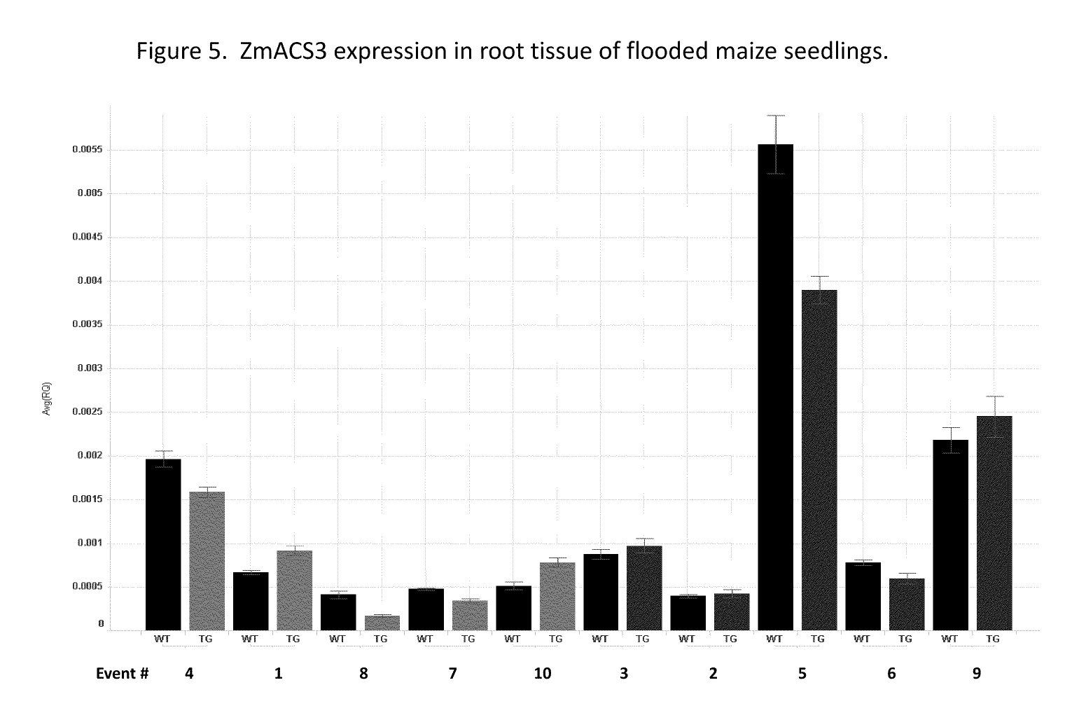

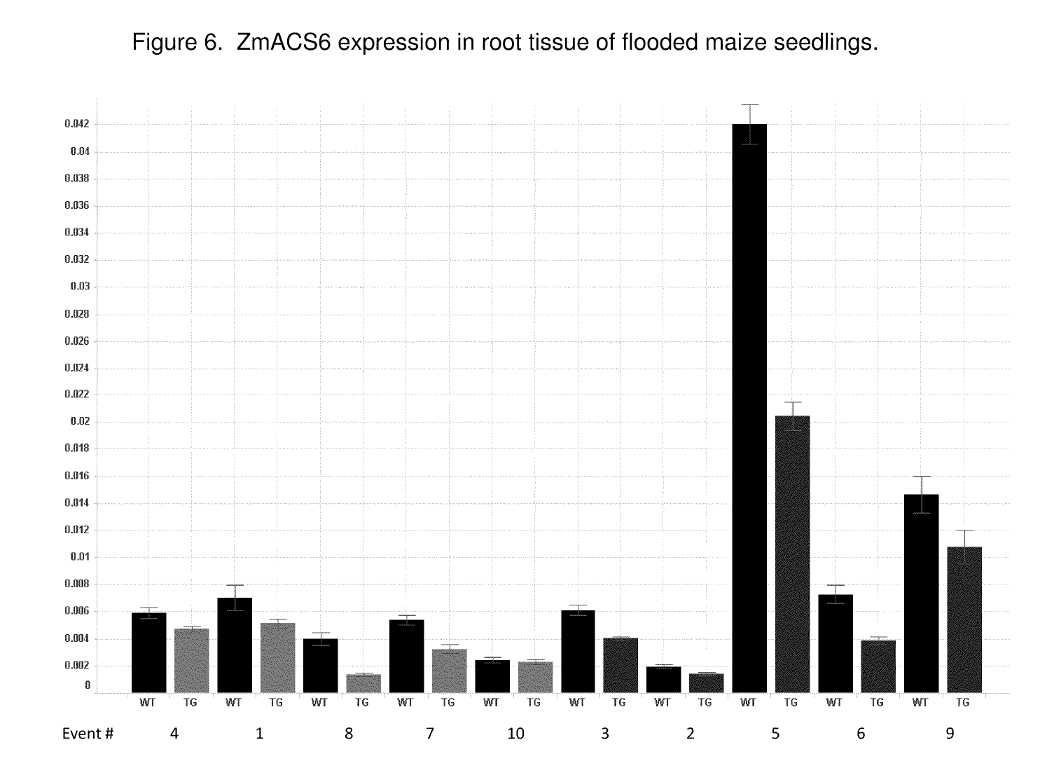

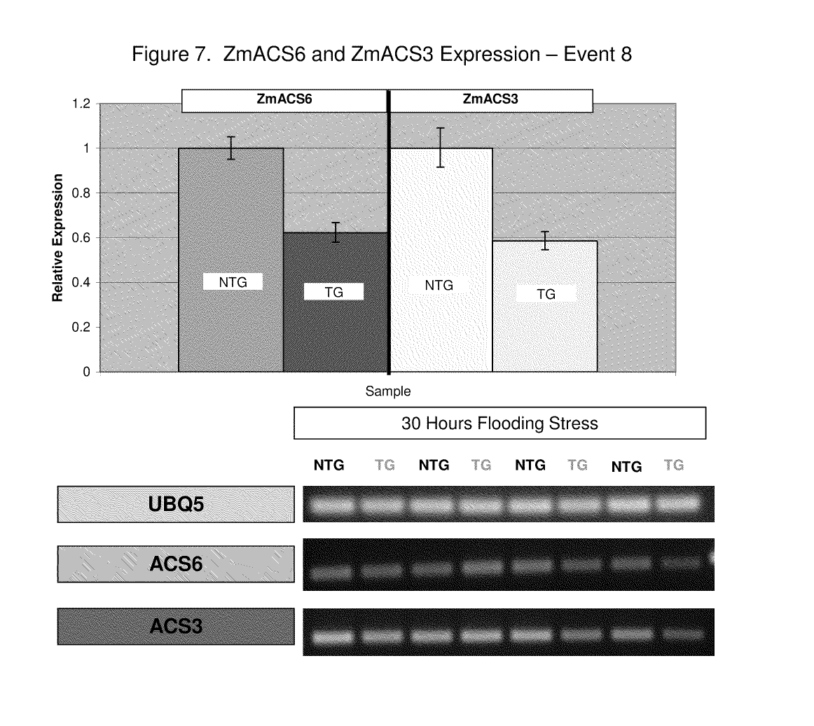

Average (n=12, comprising 4 subsamples of each of 3 plants) relative quantitation (RQ) is shown for each of 10 events in FIG. 5 for ZmACS3 and in FIG. 6 for ZmACS6. Variation in the degree of downregulation observed may result from position effect or other characteristics of independent transgenic events.

### Example 8

### Identification of Paspalum notatum ACS3

A. Preparation of cDNA Libraries and Isolation and Sequencing of cDNA Clones

An alternative method for preparation of cDNA Libraries and obtainment of sequences can be the following. mRNAs can be isolated using the Qiagen® RNA isolation kit for total RNA isolation, followed by mRNA isolation via attachment to oligo(dT) Dynabeads from Invitrogen (Life Technologies, Carlsbad, Calif.) and sequencing libraries can be prepared using the standard mRNA-Seq kit and protocol from Illumina, Inc. (San Diego, Calif.). In this method, mRNAs are fragmented using a ZnCl2 solution, reverse transcribed into cDNA using random primers, end repaired to create blunt end fragments, 3′ A-tailed, and ligated with Illumina paired-end library adaptors. Ligated cDNA fragments can then be PCR amplified using Illumina paired-end library primers, and purified PCR products can be checked for quality and quantity on the Agilent Bioanalyzer DNA 1000 chip prior to sequencing on the Genome Analyzer II equipped with a paired end module.

Reads from the sequencing runs can be soft-trimmed prior to assembly such that the first base pair of each read with an observed FASTQ quality score lower than 15 and all subsequent bases are clipped using a Python script. The Velvet assembler (Zerbino, et al., (2008) Genome Research 18:821-29) can be run under varying kmer and coverage cutoff parameters to produce several putative assemblies along a range of stringency. The contiguous sequences (contigs) within those assemblies can be combined into clusters using Vmatch software (available on the Vmatch website) such that contigs which are identified as substrings of longer contigs are grouped and eliminated, leaving a non-redundant set of longest “sentinel” contigs. These non-redundant sets can be used in alignments to homologous sequences from known model plant species.

B. Identification of cDNA Clones

In cases where the sequence assemblies are in fragments, the percent identity to other homologous genes can be used to infer which fragments represent a single gene. The fragments that appear to belong together can be computationally assembled such that a translation of the resulting nucleotide sequence will return the amino acid sequence of the homologous protein in a single open-reading frame. These computer-generated assemblies can then be aligned with other polypeptides of the invention.

### C. Genomic DNA Assemblies

Genomic sequences can be obtained using long range genomic PCR capture. Primers can be designed based on the sequence of the genomic locus and the resulting PCR product can be sequenced. The sequence can be analyzed using the FGENESH (Salamov and Solovyev, (2000) Genome Res. 10: 516-522) program, and optionally, can be aligned with homologous sequences from other species to assist in identification of putative introns.

D. Identification of Paspalum notatum ACS3.

Using the methods of Examples 8A and 8B above, an orthologue of maize ACS3 was identified in Bahia grass (Paspalum notatum). The PnACS3 nucleotide sequence is provided in SEQ ID NO: 11 and the corresponding amino acid sequence is provided in SEQ ID NO: 12. GAP analysis indicates the maize and Bahia grass coding sequences are more than 92% identical. The encoded proteins are more than 93% identical.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated polypeptide comprising an amino acid sequence selected from the group consisting of:
(a) a polypeptide comprising the amino acid sequence of SEQ ID NO: 3; and
(b) a polypeptide having at least 90% sequence identity to SEQ ID NO: 3, wherein said polypeptide has ACC Synthase 3 (ACS3) activity.

2. An isolated polynucleotide selected from the group consisting of:
(a) a polynucleotide comprising SEQ ID NO: 1 or 2;
(b) a polynucleotide encoding the amino acid sequence of SEQ ID NO: 3; and
(c) a polynucleotide having at least 90% sequence identity to SEQ ID NO: 1 or 2, wherein said polynucleotide encodes a polypeptide having ZmACS3 activity.

3. An expression cassette comprising the polynucleotide of claim 2, wherein said polynucleotide is operably linked to a promoter that drives expression in a plant.

4. A polynucleotide comprising a fragment of SEQ ID NO: 1 or SEQ ID NO: 2 in a construct which, when expressed in a plant, results in downregulation of expression of native ACS3.

5. A plant comprising a heterologous polynucleotide comprising a fragment of SEQ ID NO: 1 or SEQ ID NO: 2, wherein expression of ACS3 is downregulated relative to a control plant.

6. A plant comprising a heterologous polynucleotide of claim 4, wherein abiotic stress tolerance is increased relative to a control plant.

7. The plant of claim 6, wherein drought tolerance is increased relative to a control plant.

8. The plant of claim 5, wherein said plant comprises a plant part selected from the group consisting of a cell, a seed and a grain.

9. The plant of claim 5, wherein said plant is maize, wheat, rice, barley, sorghum or rye.

10. A method of improving drought tolerance in a plant comprising providing to said plant a polynucleotide comprising a fragment of SEQ ID NO: 1 or SEQ ID NO: 2 in a construct which, when expressed, results in downregulation of expression of native ACS3.

11. The method of claim 10 wherein the fragment is at least about 25 nucleotides in length.


SEQUENCE LISTING (12 sequences)

SEQ ID NO: 1 - DNA, 2765 bp, Zea mays
  5′UTR (214)...(372)
  exon (373)...(714)
  intron (715)...(818)
  exon (819)...(979)
  intron (980)...(1106)
  exon (1107)...(1992)
  3′UTR (1993)...(2203)
tagtagactaaagctcccggctttccctttattgttactattattatatcgtttgatttgcagcggacagtctggtcgtttccgtctgctctggaccctgccttcaattctccgccttttgacatcttgggtggtttcacacgggtatatatacttgactcgcctatatatgccaccccccacacacttggccagatcgaccaccgcatcacacagcagcagcctcttgtgctcctctctgtcctttgcttgctcatccctcgacctcgagcggcggcggcggtcactgcactgcgtgtgcgtatccatccatcttgcaggcatcgtcgtctgcaccgcgcgcttgtgcgtgagccaagggcggcagaggcgatgggtggcaagctgttgctgggcgcgagccagagccgccacgcgcacgcggtggcgtcgcctcccctgtcaaaggtggccacttccggcctccacggcgaggactcgccctacttcgccgggtggaaagcctacgacgagaacccctacgacgccgtctccaaccccggcggcgtcattcagatgggcctcgccgagaaccaggtgtccttcgacctcctcgaggggtacctcagggaccacccggaggccgcgggctggggcggctccggctccggcgtcgccagcttcagggacaacgcgctgttccaggactaccacggcctcaaggccttcagaaaggtgcgtggtgaccccgagcgcctgctctgctcgtcttgttcggtgtcctggatagaccctgcctggacaagtgcaacgtactgacggcgtcattttgcttgcaggcgatggccaacttcatggagaaggttaggggcggcaaggcccggtttgaccccgaccgcatcgtgctcaccgccggcgccacggcagctaacgagctgctcacgttcgtcctggccaacccgggagacgcgctgctgatccctactccttactatcctgggtaagcggagcgctaagaagcatccagcatgctgcatgcatgcacggacccccggcgctatgctactgttacaccgcaacagtgctctacacagacagacagacaaactgacatgctctgcttgcagtttcgacagagacctgcggtggaggacgggggtgaacatcgtgccggtgcactgcgacagcgccaacgggttccaggtcacggccgccgcgctccaggcggcgtacgaggaggccgaggcagcggggacgcgcgtccgcgccgtcctgctcaccaacccgtccaacccgctgggcaccaccgtgacgcggccggccctcgaggacgtgctcgacttcgtggcccgcaacaacatccacctcatctccgacgagatatactccggctcggtcttcgcggcgccggacctggtcagcgtggcggagctggtcgagtcccgcggcgaccccggcgtcgcggagcgcgtccacatcgtgtacagcctgtccaaggacctgggcctcccggggttccgcgtcggcgtcgtgtactcgtacaacgacgccgtggtcaccgcggcgcgccgcatgtccagcttcacgctcgtgtcgtcgcagacgcagaagacgctcgccgccatgctctcggacgccgggttcgcggacgcctacgtccgcaccaaccgccagcgcctccgggcgcggcacgaccacgtcgtcgccgggctggcccgcgcgggcgtgccgtgcctccgcggcaacgccgggctgttcgtgtggatggacatgaggcggctgctcggcgaggccaccaccgtcgccggcgagctccgcctgtgggaccggatgctgcgggaggcgaagctcaacatctcgccgggctcgtcgtgccattgctcggagcctggctggttcagggtgtgcttcgccaacatgagcctggacacgctggatgttgcactcgctaggatgagccgcttcgtagacacgtggaacaaggaaacgacagcgtcgacgcagcagcactagcagcagcagcagcatacgaagtaaattttttggagggtaaattacgtcattggacagattaaatcacagagtagttatacagggggattcttttatggtttttcgattgatggtaacatcgattttgtaacaataactatcgcctctcagatggaggagggacacatatatgtatgtatttataaaaattcttactttggcccaagcaaaagcgtctccgctacacattcagtattattgttttgcttcgtttttccctcctgagttgtggcaaacaaacacagatgacgtatgtttgctcattcattcatatatttatatctgcctggaagcgaacctaaattagcaacaagagagaacatgtccattccgagtactgagaataagtgcagcagaatgaatgctggttgttggatttaaattagatggatgttgtttgtcgtcagaaaatggagagagagaaatcagttagcgaatccctttctcgttttatactgctggttttctatacttttgaagaaaaaaaactgttttgctgaggtgttcgatgttgtaaattactgattgacttcatgtgattgcgtaacatgttactggacggaggtttacttctatacatgagtagtgtcataccaaaccaaaaaaaaaagggaatcgaaagtcttgatagcggacgtgttgattaaagaaaaagaaaccgagttccaaatgtcaacccttccttaagccggtagtaagctttgtcaaccagctaaataagggaacgcatatcc

SEQ ID NO: 2 - DNA, 1759 bp, Zea mays
  CDS (160)...(1548)
cagcagcagcctcttgtgctcctctctgtcctttgcttgctcatccctcgacctcgagcggcggcggcggtcactgcactgcgtgtgcgtatccatccatcttgcaggcatcgtcgtctgcaccgcgcgcttgtgcgtgagccaagggcggcagaggcgatgggtggcaagctgttgctgggcgcgagccagagccgccacgcgcacgcggtggcgtcgcctcccctgtcaaaggtggccacttccggcctccacggcgaggactcgccctacttcgccgggtggaaagcctacgacgagaacccctacgacgccgtctccaaccccggcggcgtcattcagatgggcctcgccgagaaccaggtgtccttcgacctcctcgaggggtacctcagggaccacccggaggccgcgggctggggcggctccggctccggcgtcgccagcttcagggacaacgcgctgttccaggactaccacggcctcaaggccttcagaaaggcgatggccaacttcatggagaaggttaggggcggcaaggcccggtttgaccccgaccgcatcgtgctcaccgccggcgccacggcagctaacgagctgctcacgttcgtcctggccaacccgggagacgcgctgctgatccctactccttactatcctggtttcgacagagacctgcggtggaggacgggggtgaacatcgtgccggtgcactgcgacagcgccaacgggttccaggtcacggccgccgcgctccaggcggcgtacgaggaggccgaggcagcggggacgcgcgtccgcgccgtcctgctcaccaacccgtccaacccgctgggcaccaccgtgacgcggccggccctcgaggacgtgctcgacttcgtggcccgcaacaacatccacctcatctccgacgagatatactccggctcggtcttcgcggcgccggacctggtcagcgtggcggagctggtcgagtcccgcggcgaccccggcgtcgcggagcgcgtccacatcgtgtacagcctgtccaaggacctgggcctcccggggttccgcgtcggcgtcgtgtactcgtacaacgacgccgtggtcaccgcggcgcgccgcatgtccagcttcacgctcgtgtcgtcgcagacgcagaagacgctcgccgccatgctctcggacgccgggttcgcggacgcctacgtccgcaccaaccgccagcgcctccgggcgcggcacgaccacgtcgtcgccgggctggcccgcgcgggcgtgccgtgcctccgcggcaacgccgggctgttcgtgtggatggacatgaggcggctgctcggcgaggccaccaccgtcgccggcgagctccgcctgtgggaccggatgctgcgggaggcgaagctcaacatctcgccgggctcgtcgtgccattgctcggagcctggctggttcagggtgtgcttcgccaacatgagcctggacacgctggatgttgcactcgctaggatgagccgcttcgtagacacgtggaacaaggaaacgacagcgtcgacgcagcagcactagcagcagcagcagcatacgaagtaaattttttggagggtaaattacgtcattggacagattaaatcacagagtagttatacagggggattcttttatggtttttcgattgatggtaacatcgattttgtaacaataactatcgcctctcagatggaggagggacacatatatgtatgtatttataaaaattcttactttggcccaagcaaaa

SEQ ID NO: 3 - protein, 462 aa, Zea mays
MGGKLLLGASQSRHAHAVASPPLSKVATSGLHGEDSPYFAGWKAYDENPYDAVSNPGGVIQMGLAENQVSFDLLEGYLRDHPEAAGWGGSGSGVASFRDNALFQDYHGLKAFRKAMANFMEKVRGGKARFDPDRIVLTAGATAANELLTFVLANPGDALLIPTPYYPGFDRDLRWRTGVNIVPVHCDSANGFQVTAAALQAAYEEAEAAGTRVRAVLLTNPSNPLGTTVTRPALEDVLDFVARNNIHLISDEIYSGSVFAAPDLVSVAELVESRGDPGVAERVHIVYSLSKDLGLPGFRVGVVYSYNDAVVTAARRMSSFTLVSSQTQKTLAAMLSDAGFADAYVRTNRQRLRARHDHVVAGLARAGVPCLRGNAGLFVWMDMRRLLGEATTVAGELRLWDRMLREAKLNISPGSSCHCSEPGWFRVCFANMSLDTLDVALARMSRFVDTWNKETTASTQQH

SEQ ID NO: 4 - protein, 482 aa, Zea mays
MIADEKPQPQLLSKKAACNSHGQDSSYFLGWEEYEKNPYDPVANPGGIIQMGLAENQLSFDLLEAWLEANPDALGLRRGGASVFRELALFQDYHGMPAFKNALARFMSEQRGYRVTFDPSNIVLTAGATSANEALMFCLADHGDAFLIPTPYYPGFDRDLKWRTGAEIVPVHCTSGNGFRLTRAALDDAYRRAQKLRLRVKGVLITNPSNPLGTTSPRADLEMLVDFVAAKGIHLVSDEIYSGTVFADPGFVSVLEVVAARAATDDGVVGVGPLSDRVHVVYSLSKDLGLPGFRVGAIYSSNAGVVSAATKMSSFGLVSSQTQHLLASLLGDRDFTRRYIAENTRRIRERREQLAEGLAAVGIECLESNAGLFCWVNMRRLMRSRSFEGEMELWKKVVFEVGLNISPGSSCHCREPGWFRVCFANMSAKTLDVALQRLGAFAEAATAGRRVLAPARSISLPVRFSWANRLTPGSAADRKAER

SEQ ID NO: 5 - DNA, 543 bp, Artificial Sequence
  segment of hp construct
cccggtacgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagagggtggtcttcgaggtggggctcaacatctccccgggctcctcctgccactgccgggagcccggctggttccgcgtctgctaaagggcgaattccagcacactggcggccgttactagtggatccgagct

SEQ ID NO: 6 - DNA, 1446 bp, Zea mays
atgatcgccgacgagaagccgcagccgcagctgctgtccaagaaggccgcctgcaacagccacggccaggactcgtcctacttcctggggtgggaggagtatgagaaaaacccatacgaccccgtcgccaaccccggcggcatcatccagatgggcctcgccgagaaccagctgtccttcgacctgctggaggcgtggctggaggccaacccggacgcgctcggcctccgccggggaggcgcctctgtattccgcgagctcgcgctcttccaggactaccacggcatgccggccttcaagaatgcattggcgaggttcatgtcggagcaacgtgggtaccgggtgaccttcgaccccagcaacatcgtgctcaccgccggagccacctcggccaacgaggccctcatgttctgcctcgccgaccacggagacgcctttctcatccccacgccatactacccagggttcgaccgtgacctcaagtggcgcaccggcgcggagatcgtccccgtgcactgcacgagcggcaacggcttccggctgacgcgcgccgcgctggacgacgcgtaccggcgcgcgcagaagctgcggctgcgcgtcaagggcgtgctcatcaccaacccttccaacccgctgggcaccacgtcgccgcgcgccgacctggagatgctggtggacttcgtggccgccaagggcatccacctggtgagcgacgagatatactcgggcacggtcttcgcggacccgggcttcgtgagcgtcctcgaggtggtggccgcgcgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagaaggtggtcttcgaggtggggctcaacatctccccgggctcctcctgccactgccgggagcccggctggttccgcgtctgcttcgccaacatgtccgccaagacgctcgacgtcgcgctccagcgcctgggcgccttcgcggaggccgccaccgcggggcgccgcgtgcttgcccccgccaggagcatcagcctcccggtccgcttcagctgggctaaccgcctcaccccgggctccgccgccgaccggaaggccgagcgg

SEQ ID NO: 7 - DNA, 8350 bp, Artificial Sequence
  ACS down-regulation expression cassette
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagcaagctggtacgattgtaatacgactcactatagggcgaattgagcgctgtttaaacgctcttcaactggaagagcggttactaccggctggatggcggggccttgatcgtgcaccgccggcgtccggataagtgactagggtcacgtgaccctagtcacttatcgagctagttaccctatgaggtgacatgaagcgctcacggttactatgacggttagcttcacgactgttggtggcagtagcgtacgacttagctatagttccggtagatctgaagttcctattccgaagttcctattcttcaaaaggtataggaacttcctcgaattgttgtggtggggtatagaggtttgatataggtggaactgctgtagagcgtggagatatagggggaaagagaacgctgatgtgacaagtgagtgagatatagggggagaaatttagggggaacgccgaacacagtctaaagaagcttgggacccaaagcactctgttcgggggtttttttttttgtctttcaactttttgctgtaatgttattcaaaataagaaaagcacttggcatggctaagaaatagagttcaacaactgaacagtacagtgtattatcaatggcataaaaaacaacccttacagcattgccgtattttattgatcaaacattcaactcaacactgacgagtggtcttccaccgatcaacggactaatgctgctttgtcagatcaccggttaagtgactagggtcacgtgaccctagtcacttaggttaccagagctggtcacctttgtccaccaagatggaactgcggccgctcattaattaagtcaggcgcgcctctagttgaagacacgttcatgtcttcatcgtaagaagacactcagtagtcttcggccagaatggccatctggattcagcaggcctagaaggccatttaaatcctgaggatctggtcttcctaaggacccgggatatcacaagtttgtacaaaaaagcaggctccggccagagttacccggaccgaagcttgcatgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccactagtaacggccgccagtgtgctggaattcgccctttagcagacgcggaaccagccgggctcccggcagtggcaggaggagcccggggagatgttgagccccacctcgaagaccaccctcttccacagctccatctcgccctcgaacgaccggctccgcatcaggcgccgcatgttgacccagcagaagagccccgcgttgctctccaggcactcgatgcccacggccgccaggccctccgccagctgctcgcgccgctccctgatccgccgcgtgttctccgcgatgtacctccgcgtgaagtccctgtcgcccaggagcgacgccaggaggtgctgcgtctgggacgacaccaggccgaagctcgacatcttggtggccgcggagaccacgccggcgttggacgagtagatggcgcccacgcggaaccccgggaggcccaggtccttggacaggctgtacaccacgtgcacgcggtccgacagcggcccaacgccgacgacgccgtcgtccgtggcggcgcgcgcggccaccacctgcagtcgacgtgcaaaggtccgccttgtttctcctctgtctcttgatctgactaatcttggtttatgattcgttgagtaattttggggaaagcttcgtccacagttttttttcgatgaacagtgccgcagtggcgctgatcttgtatgctatcctgcaatcgtggtgaacttatttcttttatatcctttactcccatgaaaaggctagtaatctttctcgatgtaacatcgtccagcactgctattaccgtgtggtccatccgacagtctggctgaacacatcatacgatctatggagcaaaaatctatcttccctgttctttaatgaaggacgtcattttcattagtatgatctaggaatgttgcaacttgcaaggaggcgtttctttctttgaatttaactaactcgttgagtggccctgtttctcggacgtaaggcctttgctgctccacacatgtccattcgaattttaccgtgtttagcaagggcgaaaagtttgcatcttgatgatttagcttgactatgcgattgctttcctggacccgtgcagctggatcccggtacgcgccgccacggacgacggcgtcgtcggcgttgggccgctgtcggaccgcgtgcacgtggtgtacagcctgtccaaggacctgggcctcccggggttccgcgtgggcgccatctactcgtccaacgccggcgtggtctccgcggccaccaagatgtcgagcttcggcctggtgtcgtcccagacgcagcacctcctggcgtcgctcctgggcgacagggacttcacgcggaggtacatcgcggagaacacgcggcggatcagggagcggcgcgagcagctggcggagggcctggcggccgtgggcatcgagtgcctggagagcaacgcggggctcttctgctgggtcaacatgcggcgcctgatgcggagccggtcgttcgagggcgagatggagctgtggaagagggtggtcttcgaggtggggctcaacatctccccgggctcctcctgccactgccgggagcccggctggttccgcgtctgctaaagggcgaattccagcacactggcggccgttactagtggatccgagctcgaattccggtccgggtcacccggtccgggcctagaaggccgatctcccgggcacccagctttcttgtacaaagtggtgatatcggaccgattaaactttaattcggtccgatgcatgtatacgaagttcctattccgaagttcctattctacatagagtataggaacttcacctggtggcgccgctagtggatcccccgggctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatgcatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgactttaacttagcctaggatccacacgacaccatgtcccccgagcgccgccccgtcgagatccgcccggccaccgccgccgacatggccgccgtgtgcgacatcgtgaaccactacatcgagacctccaccgtgaacttccgcaccgagccgcagaccccgcaggagtggatcgacgacctggagcgcctccaggaccgctacccgtggctcgtggccgaggtggagggcgtggtggccggcatcgcctacgccggcccgtggaaggcccgcaacgcctacgactggaccgtggagtccaccgtgtacgtgtcccaccgccaccagcgcctcggcctcggctccaccctctacacccacctcctcaagagcatggaggcccagggcttcaagtccgtggtggccgtgatcggcctcccgaacgacccgtccgtgcgcctccacgaggccctcggctacaccgcccgcggcaccctccgcgccgccggctacaagcacggcggctggcacgacgtcggcttctggcagcgcgacttcgagctgccggccccgccgcgcccggtgcgcccggtgacgcagatctgagtcgaaacctagacttgtccatcttctggattggccaacttaattaatgtatgaaataaaaggatgcacacatagtgacatgctaatcactataatgtgggcatcaaagttgtgtgttatgtgtaattactagttatctgaataaaagagaaagagatcatccatatttcttatcctaaatgaatgtcacgtgtctttataattctttgatgaaccagatgcatttcattaaccaaatccatatacatataaatattaatcatatataattaatatcaattgggttagcaaaacaaatctagtctaggtgtgttttgcgaattgcggccgctctagcgtatacgaagttcctattccgaagttcctattctctagaaagtataggaacttctgattccgatgacttcgtaggttcctagctcaagccgctcgtgtccaagcgtcacttacgattagctaatgattacggcatctaggaccgactagtaagtgactagggtcacgtgaccctagtcacttatacgtagaattaattcattccgattaatcgtggcctcttgctcttcaggatgaagagctatgtttaaacgtgcaagcgctactagacaattcagtacattaaaaacgtccgcaatgtgttattaagttgtctaagcgtcaatttgtttacaccacaatatatcctgccac

SEQ ID NO: 8 - DNA, 19 bp, Artificial Sequence
  primer
acgagctgctcacgttcgt

SEQ ID NO: 9 - DNA, 26 bp, Artificial Sequence
  primer
ccaggatagtaaggagtagggatcag

SEQ ID NO: 10 - DNA, 14 bp, Artificial Sequence
  MGB probe
aacccgggagacgc

SEQ ID NO: 11 - DNA, 1508 bp, Paspalum notatum
  CDS (92)...(1459)
gctgaaccgctcttccgatctcagagagtagtcttcttgcactcgtcccgtcggcgtgtgcgagggcaagcgcgaaacgaaaggagagaccatgggcggcaagctgttgctcgacgcgagccacgaggcgtcgcctcctctgtcgaaggtggccacctccggcctccacggcgaggactcgccctacttcgccgggtggaaagcctacgacgagaacccctacgacgccgtttccaacccgagcggcgtcatacagatgggcctcgccgagaaccaggtgtcgttcgacctcctggagggttacctcagggaccaccccgaggcggcgggctggggcggcgccgccgccggctccggcgtcgccagcttcagggacaacgcgctgttccaggactaccacggcctcaaggccttcaggaaggcgatggcgagcttcatggagaaaataaggggcggcaaggcgaggttcgaccccgaccgcatcgtgctcaccgccggcgcgacggcggccaacgagctgctcacgttcatcctggccaacccgggagacgcgctgctgatccctacgccttactaccctggtttcgacagagacttgaggtggaggacgggggtgaacatcgtgccggtgcactgcgacagcgccaacgggttccaggtcacggtcgccgcgctgcaggcggcgtacgaggaggccgaggccgcggggatgcgcgtccgcgccgtcctgctcaccaacccgtccaacccgctgggcaccaccgtgaagcgggcggtgctcgaggacgtgctcgacttcgtggcgcgcaacaacatccacctcatctccgacgagatctactccggctcggtgttcgcggcgccggacctggtcagcgtggcggagctcgtcgagtcccgcggcgacgccggcgtcgccgcccgcgtccacatcgtgtacagcctgtccaaggacctgggcctcccgggcttccgcgtcggcgtggtgtactcgtacaacgacgccgtggtcaccacggcgcgccgcatgtccagcttcacgctcgtgtcgtcgcagacgcagaagacgctcgccgccatgctctccgacgcgggcttcgccgacgcctacatccgcaccaaccgcgagcgcctccgggcgcggcacgaccacgtcgtggccgggctggcccgcgccggcgtcccgtgcctgcccggcaatgccggcctgttcgtgtggatggacatgcggcggctgctcggcggcgaggccaccgtcgccggcgagctcaggctgtgggaccggatgctgcgggaggtgaagctcaacatctcgccgggatcgtcgtgccattgctccgaggccggctggttcagggtgtgcttcgctaacatgagcctggacacgctggacgttgcactcgcaaggatgaaccgcttcatggacaagtggagcatcaaggaaacaacacaacagtagtgtcaacgcaacaagaacaacattagaggatcgagtcaaaaagtttttt

SEQ ID NO: 12 - protein, 455 aa, Paspalum notatum
MGGKLLLDASHEASPPLSKVATSGLHGEDSPYFAGWKAYDENPYDAVSNPSGVIQMGLAENQVSFDLLEGYLRDHPEAAGWGGAAAGSGVASFRDNALFQDYHGLKAFRKAMASFMEKIRGGKARFDPDRIVLTAGATAANELLTFILANPGDALLIPTPYYPGFDRDLRWRTGVNIVPVHCDSANGFQVTVAALQAAYEEAEAAGMRVRAVLLTNPSNPLGTTVKRAVLEDVLDFVARNNIHLISDEIYSGSVFAAPDLVSVAELVESRGDAGVAARVHIVYSLSKDLGLPGFRVGVVYSYNDAVVTTARRMSSFTLVSSQTQKTLAAMLSDAGFADAYIRTNRERLRARHDHVVAGLARAGVPCLPGNAGLFVWMDMRRLLGGEATVAGELRLWDRMLREVKLNISPGSSCHCSEAGWFRVCFANMSLDTLDVALARMNRFMDKWSIKETTQQ